(19) United States
(12) Patent Application Publication
(10) Pub. No.: US 2025/0099227 A1
(43) Pub. Date: Mar. 27, 2025

(54) Volumizing Grafts Suitable for Plastic and Reconstructive Surgery

(71) Applicant: Musculoskeletal Transplant Foundation, Edison, NJ (US)

(72) Inventors: Juliana Gullotta, Ossinig, NY (US); Evangalia Chnari, Scotch Plains, NJ (US); Marc Long, Monmouth Junction, NJ (US)

(21) Appl. No.: 18/832,763

(22) PCT Filed: Jan. 24, 2023
(86) PCT No.: PCT/US2023/011405

§ 371 Date: Jul. 24, 2024

Related U.S. Application Data
(60) Provisional application No. 63/302,777, filed on Jan. 25, 2022.

(51) Int. Cl. A61F 2/12; A61L 27/36; A61L 27/38; A61L 27/54; A61L 27/56

(52) U.S. Cl. CPC A61F 2/12; A61L 27/3608; A61L 27/3612; A61L 27/3629; A61L 27/3641; A61L 27/38; A61L 27/54; A61L 27/56; A61F 2210/0076; A61F 2220/0075; A61F 2230/0071; A61L 2430/04

(57) ABSTRACT

Volumizing grafts useful for plastic and reconstructive surgery, especially those involving replacement or enhancement of tissue mass and volume. The volumizing graft includes a sheet shaped body made of a biocompatible material which forms, or is capable of forming, a shape having a cavity for receiving, holding, containing, etc., one or more additional components. Either or both of the sheet shaped body and additional components may be bioactive. Either or both of the sheet shaped body and additional components may comprise tissue derived matrices. The sheet shaped body of the volumizing graft enables the additional components to be positioned, oriented, affixed, or some combination thereof, in or proximate to a treatment site, either permanently or temporarily. This may allow subsequent removal, repositioning, replacement, supplementing, or a combination thereof, of the additional components, at the treatment site.

### CROSS-REFERENCE TO RELATED APPLICATIONS

The present application is a national stage of International Patent Application No. PCT/US2023/011405, filed Jan. 24, 2023, which claims the benefit of U.S. Provisional Application No. 63/302,777, filed Jan. 25, 2022, the entire disclosures of both of which are incorporated by reference herein.

### FIELD

The present invention relates generally to volumizing grafts suitable for plastic and reconstructive surgery. More particularly, volumizing grafts which may comprise a tissue derived matrix and include a body with a cavity for receiving and containing additional components therein are suitable and beneficial for use in plastic and reconstructive surgeries which involve or require replacement or augmentation of tissue mass and volume.

### BACKGROUND

Plastic and reconstructive surgeries are very common in medicine today. There are many reasons why such surgical procedures are performed including the repair, reconstruction, modification, etc., of one or more body features which are damaged or would benefit from reconstruction, for functional or aesthetic reasons, or both. Repair and reconstruction of one or more body features may be necessitated or desired for any of several reason including, but not limited to, disease, trauma, atrophy, malfunction, lack of full function, appearance, aesthetic concerns or preferences, as well as for repair, reconstruction, or both, in connection with other treatments or procedures (whether surgical, or otherwise).

Grafts and implants of many different types, configurations, and properties have been developed for use in plastic and reconstructive surgeries. Such grafts and implants may be made of natural materials, synthetic materials, and are sometimes combinations of natural and synthetic materials. Sometimes grafts and implants are used together, concurrently or at different times, to treat the same body feature(s). Notwithstanding the wide variety of grafts and implants currently available, many of which are adapted and customized for use in specific plastic and reconstructive surgeries, there remain issues and concerns relating to the efficacy, degree of success, longevity, shortcomings, side effects, etc., which are expected and provided by the grafts and implants developed to date.

Grafts described and contemplated hereinbelow address several of the issues and concerns which arise when many of the currently available grafts are used for repair and reconstruction surgery.

### SUMMARY

The present invention relates generally to tissue-derived grafts suitable for plastic and reconstructive surgery. More particularly, the tissue-derived grafts are suitable and beneficial for use in plastic and reconstructive surgeries which involve or require replacement or enhancement of tissue mass and volume.

[symbol]

### DETAILED DESCRIPTION

Detailed embodiments of the present invention are disclosed herein. It should be understood that the disclosed embodiments are merely illustrative of the invention which may be embodied in various forms. In addition, each of the examples given in connection with the various embodiments of the invention is intended to be illustrative, and not restrictive. Further, the figures are not necessarily to scale, and some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as examples for teaching one skilled in the art to variously employ the present invention.

The following definitions are provided for clarification and enhanced understanding of the following description and are not intended to be limiting.

As used herein, the term “autologous” means a material or substance which is derived or transferred from the same recipient into or onto which it is later implanted.

As used herein, the term “bioactive” means a substance or material which has a physiological effect on a recipient of the bioactive substance or material, where the physiological effect is often, but need not be, an intended component of treating of a condition. Physiological effects of bioactive materials and substances comprise, but are not limited to, one or more of inducing, participating in, facilitating, or otherwise enhancing biological processes such as, without limitation, healing, generating, regenerating, repairing and reconstructing tissue, an organ, or a body feature, affected by a condition to be treated. Bioactive materials and substances may be administered to a recipient in any suitable and effective manner, whether directly or indirectly to the tissue, organ, or body feature to be treated. One or more bioactive materials or substances may be administered to a recipient separately (alone) or in combination with other substances, materials, or devices.

The term “biocompatible,” is used herein to describe materials, substances, grafts, implants and other devices which, when implanted in a living recipient, does not cause one or more adverse effects such as, without limitation, toxicity, injury, foreign body reaction or rejection, irritation, allergic or other histamine reaction, disruption of cellular structure of function, etc. A biocompatible material may, but does not have to, be bioactive.

As used herein, the term “carrier” means a substance or material capable of being mixed. with another material, which may be in any physical state (solid, liquid, gel, etc.) and may be suspension, dispersion, solution, etc.) and is generally, but not required to be, inert or unreactive. In the present context of grafts and implants, carriers added to or combined with materials or matrices which form a graft or implant should generally be biocompatible, and may or may not be dissolvable, resorbable, etc. One or more carrier materials may be added or included at any point or step during methods for processing one or more tissue samples to produce the particulate tissue-derived matrix or the volumizing graft described and contemplated herein. Additionally, one or more carrier materials may be added at or near the end of such methods, or even by a medical practitioner at the point of use, such as during a surgical, minimally invasive, superficial, or surface treatment procedure, to form a paste, a gel, a slurry, or a suspension. Suitable biocompatible carriers include, without limitation, an isotonic solution, a sodium chloride solution, lactated Ringer's solution, a phosphate-buffered saline solution, platelet rich plasma (PRP), hyaluronic acid, a derivative of hyaluronic acid, sodium hyaluronate, cellulose, carboxymethyl cellulose, thrombin, fibrin, glycerin, collagen, lecithin, sugars, polysaccharides, and solutions thereof. Additional suitable biocompatible carriers are natural polymers and. synthetic polymers including, without limitation, polysaccharides, nucleic acids, carbohydrates, proteins, polypeptides, poly(.alpha.-hydroxy acids), poly(lactones), poly(amino acids), poly(anhydrides), poly(orthoesters), poly(anhydride-co-imides), poly(orthocarbonates), poly(.alpha.-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(.epsilon.-caprolactone), poly(.delta.-valerolactone), poly(.gamma.-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate) polyamide, and copolymers thereof. Suitable biocompatible carriers also include one or more autologous materials such as, without limitation, whole blood, blood components, PRP, lipoaspirate adipose, adipose stromal vascular fraction (SVF), bone marrow, BMAC, autologous cells derived from bone marrow, blood, adipose, other autologous tissue, and combinations thereof.

As used herein, the term “condition” refers to a variety of healthy and unhealthy states of a living subject and its organs, tissues, and biological systems, and includes both normal, healthy and functioning states, as well as abnormal, unhealthy, damaged, and malfunctioning states, whether caused by a disorder, disease, infection, injury, trauma, or other mechanism or event.

The term “contacting” and “implanting” and their various grammatical forms, when used herein in connection with placement or positioning of grafts and implants, are used herein interchangeably and refer to a state or condition of touching or of immediate or local proximity, internally or externally, such as positioning or implanting a composition, graft, or implant in, on, or proximate to, a body feature to be treated. Contacting a composition to a target destination, such as a body feature or an implant site, may occur by any means of administration known to persons of ordinary skill in the relevant art. When used in connection with steps and methods for producing grafts and implants, the term “contacting” and its various grammatical forms means at least partial physical contact between two or more components, ingredients, solutions, mixtures, treatment agents, reaction agents, preservation agents, etc., and combinations thereof, for some period of time greater than zero seconds. Where the temperature and/or pressure at which contacting occurs are unspecified, it should be assumed that the temperature is ambient or room temperature, and the pressure is normal atmospheric pressure at sea level and ambient or room temperature.

The terms “derived,” “derived from,” and “produced from” and all their grammatical forms, are used herein interchangeably to mean that the material or composition being described is produced by subjecting one or more specified starting materials to one or more process steps or techniques, which may be physical, chemical, or combinations of physical and chemical techniques, as are appropriate depending on the starting material and intended material or composition product. For example, without limitation, a “tissue derived matrix” is a composition which has been produced by subjecting a selected tissue sample to one or more process steps or techniques, which may be physical, chemical, or combinations thereof. (e.g., recovering the tissue sample from a donor, cutting to size, disinfecting, decellularizing, and drying by lyophilizing). A component of a graft which is produced from a biocompatible synthetic polymer means that the component is produced by subjecting the selected biocompatible synthetic polymer to one or more process steps or techniques, which may be physical, chemical, or combinations thereof (e.g., optionally blending, combining, mixing, etc., the selected polymer with one or more additives such as rheology modifiers, emulsifiers, dispersing agents, stabilizers, etc., and shaping by thermoforming, extrusion, crosslinking, etc.).

As used herein, the term “donor” means a living or deceased mammal from which one or more tissues, organs, other body parts, or portions thereof, are recovered or harvested. Suitable mammalian donors include, but are not limited to: human, primate, bovine, porcine, equine, ovine, rodent, leporine, canine, feline, etc.

As used herein, the term “graft” means a biologically compatible material, tissue, or substance which is introduced into the body of a subject, either permanently or temporarily, to replace, improve or supplement the structure or function of tissue, an organ, or other body feature, of a subject or patient, or to improve, retain, support, position, or supplement an implant or another graft. Grafts may be absorbed, integrated, remodeled, etc., in whole or in part, into a patient's body after implantation. A graft may also be configured and used for the administration or delivery of a therapeutic agent or other bioactive substance.

The terms “growth-inductive components” or “growth-inductive factors” or “tissuegenic factors” are used interchangeably to refer to the plethora of mediators associated with tissue development and repair. Some, but by no means all, examples of such components and factors include, without limitation, adiponectin, bone morphogenic proteins (BMPs), vascular endothelial growth factor (VEGF), basic fibroblast growth factor (bFGF), transforming growth factor beta (TGFβ), and platelet-derived growth factor (PDGF), tumor necrosis factor alpha (TNF-α), prostaglandins, interleukins, etc.

The term “implant,” as used herein, means a device or material that replaces a missing body feature or portion thereof, or supplements, reshapes, or creates a desired body feature. Replacing, supplementing, reshaping, and creating a desired body feature may be necessary or desired for any of several possible reasons including, without limitation, trauma, disease, congenital condition, aesthetic preference, or a combination there, and is intended to provide or restore the normal or intended function(s) and/or shape of the body feature. An implant may be simple, such as being monolithic or having a recognizable geometric shape, or complex, such as having several features, components, shapes, protrusions, cavities, etc., depending on the intended use. Furthermore, an implant can be any material, device or substance which is introduced into the body of a subject, either permanently or temporarily, to replace, improve or supplement the structure or function of tissue, an organ, or other body feature of the subject and includes, but is not limited to, those used for the administration or delivery of a therapeutic agent or substance. A “prosthetic device” refers to a particular kind of implant and means a device, either external or internal, that substitutes for or supplements a missing or defective part of the body (i.e., body feature), or augments performance of a natural function of a body feature.

The term “isolating” and its various grammatical forms as used herein refers to placing, setting apart, or obtaining a protein, molecule, substance, nucleic acid, peptide, cell or particle, in a form essentially free from contaminants or other materials with which it is commonly associated, separate from its natural environment.

The term “particulate,” as used herein, means a physical form comprising a plurality of particles, pieces, or combinations thereof, wherein the size (average diameter) of the particles and/or pieces range in size from about 0.001 millimeters (mm) to about 10 centimeters (cm).

“Sterilization” and “sterilizing” as used herein, in all of their grammatical forms, is any process that renders an object (e.g., a tissue, a retainer for tissue, or an implement for processing tissue) essentially free from pathogenic organisms and/or viruses by destroying them or otherwise inhibiting their growth or vital activity. Such processes may include exposure of the object to one or more, without limitation, of gamma radiation, electron beam radiation, chemical agents (e.g., alcohol, phenol, ethylene oxide gas, acids, bases, or peroxides), heat, or ultraviolet radiation for sufficient duration and dosages. When sterilization is performed on a finished tissue product in its final packaging, the process may be referred to as “terminal sterilization”.

As used herein, the term “tissue derived matrix” means a material which is produced, by one or more processing steps and techniques, from one or more samples of tissue recovered from one or more donors. The one or more processing steps and techniques may be physical, chemical, and combinations thereof. The samples of tissue may be the same or different types of tissue as one another. Furthermore, it is possible for a single tissue sample to include more than one type of tissue when recovered, and may or may not be subjected to separation of the different types of tissue during processing. The donor of one or more of the tissue samples from which a tissue derived matrix is produced may be: the same individual as the recipient of a graft comprising the tissue derived matrix (i.e., autogenic), or a different individual of the same species as the recipient (i.e., allogenic), or a different species than the recipient (i.e., xenogenic).

The invention described and contemplated herein provides a volumizing graft suitable and useful for implanting in a treatment site of a subject or patient during reconstructive or cosmetic surgical procedures. As will be described in further detail later, the volumizing grafts described and contemplated herein provide the ability to reconstruct or reshape body features which include or are proximate to the treatment site and which, for whatever reason, involve the replacement, addition, generation, or formation of a relatively large volume (as well as smaller volumes), of tissue at the treatment site which would be proximate to a body feature being treated. For example, without limitation, the volumizing graft according to the invention described and contemplated herein might be useful for reconstruction and augmentation or replacement of breast tissue which has been removed during partial or full mastectomy procedures, respectively. Of course, persons of ordinary skill and practitioners in the relevant art will recognize many treatment and procedures for which the volumizing grafts described herein will be useful and may provide improved benefits and results compared to presently available graft materials.

The volumizing graft generally comprises a sheet shaped body, which has a first surface and a second surface and forms, or is capable of forming, a shape having a cavity which is at least partially defined by the first surface. The volumizing graft further includes an external surface which is at least partially formed by the second surface of the sheet shaped body and which, after implanting, faces and may contact host tissue at the treatment site.

It is contemplated that the volumizing graft may be provided at least partially in a shape having the aforesaid cavity, or it may be provided as a sheet or substantially planar element having sufficient flexibility to allow it to be reshaped to form the cavity defined by the first surface. For example, without limitation, the volumizing graft may have the shape of pouch, sling, balloon, wrap, cap, covering, container, retainer, envelope, or any other shape which includes the aforesaid cavity. The cavity receives and retains, for at least a period of time, therein an additional component or components (i.e., one or more additional components), as described in further detail hereinbelow. The sheet shaped body of the volumizing graft described and contemplated herein enables one or more additional components to be positioned, oriented, affixed, or some combination thereof, in or proximate a treatment site, cither permanently or temporarily, and optionally with the ability to subsequently remove, reposition, or both, at least a portion of the additional component, or to replace, supplement, or both, at least a portion of the additional component at a treatment site. Additionally, while the one or more additional components may be bioactive, it is contemplated that the sheet shaped body may comprise one or more sheet shaped components which are bioactive.

The sheet shaped body comprises one or more sheet shaped components, each comprising a biocompatible material. In some embodiments, the sheet shaped body may comprise two or more sheet shaped components which are attached, connected, layered, arranged, affixed, or otherwise combined, with one another to form the sheet shaped body having a first surface which comprises one or more surfaces of the two or more sheet shaped components and which defines the cavity.

In some embodiments, the sheet shaped body comprises first and second sheet shaped components which are layered together and overlap one another. In such embodiments, the first surface of the sheet shaped body may include the surface of only one of the first or second sheet shaped components, such as, without limitation, when the first and second sheet shaped components are coextensive and completely overlap one another. One the other hand, where the first and second sheet shaped components are attached along at least a portion of their edges, or are layered only partially overlapping one another, it is possible and contemplated that the first surface of the sheet shaped body may include portions, or even substantially all, of the surfaces of both of the first or second sheet shaped components.

The cavity of the volumizing graft is sized and shaped to at least partially cover and contain the additional component therein, or even to fully cover and contain the one or more additional components therein. Additionally, it is contemplated that the additional component is retained in the cavity for at least some period of time, which may be relatively short, such as from minutes or an hour, or longer such as up to several days, weeks, or months, such as when the additional component is dissolvable, absorbable, or otherwise capable of remodeling or integrating, with the tissue of a treatment site.

As will be recognized and is determinable by persons of ordinary skill in the relevant art, the degree to which the one or more additional components are covered and contained within the cavity will depend on several factors and conditions including, but not limited to, the physical form and characteristics of the additional component (e.g., particulate or monolithic, flowable or not, etc.) and the particular shape, size, and features of the sheet shaped body of the retainer graft. For example, the sheet shaped body may have a size which is sufficient to cover all, or only a portion, of the additional component.

In some embodiments, such as those in which the sheet shaped body of the volumizing graft is not large enough to effectively cover and contain (i.e., without unintended movement, shifting, or reorienting) one or more additional components, the sheet shaped body may also include one or more attachment features to facilitate wrapping and securing the body around the additional component or components. Such attachment features are not particularly limited and include features such as tabs, straps, ribbons, or other extensions, for wrapping around and retaining the additional component in the cavity defined by the first surface of the body. Other kinds of attachment features may be included such as, without limitation, one or more sutures already inserted through the body, or one or more apertures through the body for receiving sutures therethrough to facilitate wrapping and securing the volumizing graft around the additional component.

According to some embodiments, the sheet shaped body, or one or more sheet shaped components from which it is formed, may include one or more pass-through features which allow passage or migration of fluids, bioactive substances, cells, regenerating tissue, etc. therethrough. Similarly, the sheet shaped body, or one or more sheet shaped components from which it is formed, may include, or one or more expansion features which allow the sheet shaped body or sheet shaped component(s) to expand and cover or contain more of an additional component held in the cavity, more of a treatment site, or a combination thereof. Some examples of features suitable to allow pass-through, expansion, or both, include, without limitation, perforations, openings, slots, meshing, grooves, channels, etc., and combinations thereof. Suitable pass-through features typically, but do not have to, extend entirely through a sheet shaped body or component from a first surface to a second surface thereof.

It is contemplated that the sheet shaped body of the volumizing grafts may be shaped or configured in any of several potentially adaptable and beneficial ways. For example, without limitation, the sheet shaped body may, prior to reshaping to have a cavity, further have a flower or lotus-like design with petals or tabs extending outward in several directions from a center of the body, where the center area would support a mass or quantity of tissue derived matrix (e.g., adipose or other tissue type) and the petals or tabs would fold over and enclose the mass or quantity tissue derived matrix. In another embodiment, the sheet shaped body may include two or more sheet shaped components which are layered with one another and each of which comprises a tissue derived matrix which may or may not be the same as the others. For example without limitation, a first sheet shaped component may comprise dermal tissue derived matrix and a second sheet shaped component may comprise adipose tissue derived matrix and be layered or overlapped onto the first sheet shaped component. Additionally, it is contemplated that a sheet shaped body may have partial thickness meshing (i.e., meshing which extends only partially through the thickness of the body) or may comprise two or more sheet shaped components of which an outermost positioned sheet shaped component is meshed to allow greater expansion than inner unmeshed or thicker sheet shaped components of the body. Such partial thickness meshing, or outermost meshed sheet shaped components, for the sheet shaped body of a volumizing graft could provide both expansion capability and greater potential for ingrowth or migration of cells and new tissue growth through an outer portion of the body, while maintaining greater tensile strength at the first surface which defines the cavity to better support an additional component (e.g., comprising a sponge-like tissue derived matrix) disposed within the cavity of the body.

As already mentioned, each of the one or more sheet shaped components which form the sheet shaped body of the volumizing graft comprises is made from a biocompatible material. Where the sheet shaped body includes two or more combined sheet shaped components, the biocompatible materials of each of the sheet shaped components may be the same or different from one another. It is further contemplated that the sheet shaped body may include auxiliary components, whether sheet shaped or not, which may or may not comprise biocompatible materials, but such auxiliary components should be removed or separated from the volumizing graft prior to or during implanting at a treatment site.

The biocompatible material may comprise a natural material, a synthetic material, or a combination thereof. Suitable natural materials include, for example without limitation, woven or non-woven textiles made from cotton, silk, etc., tissue derived matrices, and combinations thereof. Suitable synthetic materials include, for example without limitation, woven or nonwoven textiles or meshes made from nylon, polyesters, polypropylene, polyglycolic acid, glycolide, lactide, trimethylene carbonate, and the like, as well as copolymers and other combinations thereof.

Tissue derived matrices are produced by subjecting one or more tissue samples to one or more processing techniques to provide one or more sheets of tissue-derived matrix having desired characteristics. Tissue derived matrices are biocompatible and may or may not also be bioactive. Tissue-derived matrices suitable to form sheet shaped components for making a sheet shaped body of a volumizing graft described and contemplated herein are not particularly limited, except that the tissue derived matrix must be sheet shaped or capable of being formed into a sheet (i.e., a substantially, but not necessarily exactly, planar element capable of forming a shape having a cavity as described above). In some embodiments of the volumizing graft in which the sheet shaped body comprises at least one tissue derived matrix, one or more of the tissue derived. matrices may contain extracellular matrix with its natural structure remaining substantially intact and not disrupted so the resulting volumizing graft will provide the desired degree of support and structural integrity when implanted.

Tissue samples processed to produce sheet shaped components of the sheet shaped body of the volumizing grafts are recovered from one or more donors and may comprise any one or more tissue types which have a generally planar shape including, without limitation: an adipose tissue, a connective tissue, a dermis tissue, a duodenal tissue, a fascia tissue, an intestinal mucosal tissue, an intestinal serosal tissue, a membranous tissue, a muscle tissue, a pericardial tissue, a periosteal tissue, a peritoneal tissue, placental tissue and combinations thereof.

Each of the one or more tissue samples used to produce the particulate tissue-derived matrix may comprise one or more tissue types selected from the list provided above. In some embodiments, for example without limitation, at least one of the one or more tissue samples comprises an adipose tissue, a fascial tissue, a muscle tissue or a dermal tissue. As will be recognized by persons of ordinary skill in the relevant art, it is not uncommon for a tissue sample to sometimes include more than one tissue type when recovered from a donor. For example, without limitation, a tissue sample comprising adipose tissue may also comprise one or more tissue types selected from dermal tissue, fascial tissue, and muscle tissue. As another example, without limitation, a tissue sample comprising cancellous bone tissue may also comprise one or more of cortical bone tissue, cartilage tissue, and muscle tissue. Furthermore, the tissue types included in a tissue sample which comprises multiple types of tissue may, but do not have to be, arranged in their original native configuration when recovered from one or more donors or when subjected to processing which produces a tissue derived matrix.

Donors from which the one or more tissue samples are recovered are mammalian and include, without limitation, humans, primates, bovines, porcines, equines, ovines, rodents, leporines, canines, felines, and combinations thereof. Moreover, it is possible for a tissue sample and, therefore, the tissue-derived matrix produced therefrom, as well as a volumizing graft comprising the tissue-derived matrix, to be autogenic, allogenic, xenogenic, or a combination thereof, with respect to the recipient of the matrix or graft. The one or more donors may be living, cadaveric, or a combination thereof.

Methods for producing the volumizing grafts described and contemplated herein comprise providing or producing a sheet shaped body having a first surface and a second surface and formed into, or being capable of forming, a shape having a cavity which is at least partially defined by the first surface. In some embodiments, the sheet shaped body may be produced by obtaining or producing one or more sheet shaped components, each comprising a biocompatible material, and then subjecting the one or more sheet shaped components to one or more physical processes, chemical processes, or combinations thereof, to produce the sheet shaped body having suitable and desired size, shape, and other characteristics for implanting at a treatment site.

The particular physical and chemical processes applied to the biocompatible materials to form the sheet shaped components and sheet shaped body are not particularly limited and will depend upon what type of biocompatible material is used. For example, without limitation, biocompatible material such as natural or synthetic polymer may be obtained already in the form of one or more sheets suitable for reshaping into a shape having a cavity for surrounding and containing one or more additional components.

Where two or more sheet shaped components are used, they may be combined together to form a single sheet shaped body as described above (i.e., by attaching, connecting, layering, arranging, affixing, and other combining techniques). For example, without limitation, effective and suitable combining techniques may include one or more of: suturing, stapling, adhering, crosslinking, covalent or ionic bonding, layering or arranging followed by dehydrating, etc. Suitable biocompatible materials include, without limitation, natural materials, synthetic materials, and combinations thereof, which are capable of being shaped into a sheet or sufficiently planar element, and then into a shape having a cavity for receiving and retaining one or more additional components, as described above.

Methods for producing the presently described and contemplated volumizing grafts may further comprise reshaping, resizing, or both, the sheet shaped body to a shape having the aforesaid cavity, where the reshaping may be performed during or after producing the sheet shaped body from natural materials, synthetic materials, or both. Such reshaping and resizing may, for example without limitation, be performed using one or more techniques including: folding, fastening, adhering, cutting, stamping, layering, molding, folding, curling, suturing, sewing, extruding, manipulating, additive (3D) printing, and other shaping and resizing techniques, and combinations thereof.

In some embodiments where the volumizing graft will comprise one or more tissue derived matrices either in the sheet shaped body or in a sheet shaped component used to form the sheet shaped body, the method for producing the volumizing grafts may comprise obtaining or producing a tissue derived matrix and subjecting that tissue derived matrix to one or more physical processes, chemical processes, or both, to produce the sheet shaped component or sheet shaped body of the graft. The method may further comprise adding, mixing, or otherwise combining additional tissue derived matrices, sheet shaped components, or other materials or substances, during production of the sheet shaped component, the sheet shaped body, or both.

Accordingly, in such embodiments, the method for producing volumizing grafts having a sheet shaped body which includes at least one tissue derived matrix, may comprise one or more of the following steps:
(a) isolating a sample of a tissue having a generally planar shape from its source;
(b) pre-processing each of the one or more tissue samples;
(c) optionally, delipidizing one or more of the one or more tissue samples;
(d) optionally, decellularizing one or more of the one or more tissue samples;
(e) optionally, disinfecting one or more of the one or more tissue samples; and
(f) post-processing the one or more tissue samples.

In various embodiments of the disclosed method, some of the aforesaid steps may be omitted, the order of the steps may be varied, or additional steps may be provided. The “one or more tissue samples” and the “tissue sample(s)” of the method may be the tissue sample(s) in the form isolated from the source, the pre-processed tissue sample(s), the delipidized tissue sample(s), the decellularized (or acellular) tissue sample(s), the disinfected tissue sample(s), the post-processed tissue sample(s), or the packaged tissue sample(s). Embodiments of the disclosed method are discussed further hereinbelow, and exemplary embodiments are presented.

According to some embodiments, isolating step (a) comprises excising one or more tissue samples having a generally planar shape and comprising at least one desired tissue type from their source(s). The source(s) of the one or more tissue samples are as described above, i.e., one or more mammals. According to some embodiments, isolating step (a) comprises removing the tissue sample(s) comprising desired tissue type(s) from its source. According to some embodiments, isolating step (a) comprises aspirating the tissue sample(s) comprising desired tissue type(s) from its source. According to some embodiments, isolating step (a) comprises recovering the tissue sample(s) comprising desired tissue type(s) from its source, According to some embodiments, isolating step (a) comprises dissecting the tissue sample(s) comprising desired tissue type(s) from its source. In some embodiments, the source of the tissue sample(s) comprising desired tissue type(s) is a frozen source and the isolating step (a) includes thawing the source.

According to some embodiments, isolating step (a) comprises separating the tissue sample(s) which comprises the desired tissue type(s) from adjacent tissues of a different tissue type than the desired tissue type(s). According to some embodiments, isolating step (a) comprises cutting the adjacent tissues from the tissue sample(s). According to some embodiments, isolating step (a) comprises pulling the adjacent tissues away from the tissue sample(s). According to some embodiments, isolating step (a) comprises scraping the adjacent tissues from the tissue sample(s). According to some embodiments, isolating step (a) comprises separating the adjacent tissues from the tissue sample(s) by differential settling of the desired tissue type(s) and adjacent tissues in a liquid medium.

As described above, suitable tissue types and combinations thereof in each tissue sample include any one or more tissue types which have a generally planar shape including, without limitation: an adipose tissue, a connective tissue, a dermis tissue, a duodenal tissue, a fascia tissue, an intestinal mucosal tissue, an intestinal serosal tissue, a membranous tissue, a muscle tissue, a pericardial tissue, a periosteal tissue, a peritoneal tissue, and combinations thereof.

According to some embodiments, isolating step (a) is performed at a temperature of about 25° C. According to some embodiments, isolating step (a) is performed at a temperature of about 4° C. to about 10° C. According to some embodiments, isolating step (a) is performed at an ambient temperature.

According to some embodiments, isolating step (a) comprises reducing the bioburden of the tissue sample(s) before performing the isolating step (a). According to some embodiments, isolating step (a) comprises rinsing with a liquid prior to reduce bioburden levels on the surface of the tissue sample(s). According to some embodiments, the liquid comprises phosphate buffered saline (PBS). According to some embodiments, the liquid comprises acetic acid. According to some embodiments, the liquid comprises peracetic acid. According to some embodiments, the liquid comprises hydrogen peroxide.

According to some embodiments, pre-processing step (b) includes size reduction of the tissue sample(s). According to some embodiments, pre-processing step (b) comprises cutting or slicing the tissue sample(s). According to some embodiments, pre-processing step (b) comprises cutting the tissue sample(s) into chunks. According to some embodiments, pre-processing step (b) comprises mincing the tissue sample(s). According to some embodiments, pre-processing step (b) comprises grinding the tissue sample(s). According to some embodiments, pre-processing step (b) comprises milling the tissue sample(s). According to some embodiments, pre-processing step (b) comprises freezer milling the tissue sample(s). According to some embodiments, pre-processing step (b) comprises homogenizing the tissue sample(s).

According to some embodiments, pre-processing step (b) comprises separating components of the tissue sample(s) by differential settling of the components of the tissue sample(s). According to some embodiments, pre-processing step (b) comprises separating components of the tissue sample(s) by centrifuging. According to some embodiments, pre-processing step (b) comprises separating components of the tissue sample(s) by filtering. As will be recognized and understood by persons of ordinary skill in the relevant art, adjusting the parameters of any of the techniques used to perform pre-processing step (b), for example without limitation, grinding, milling, centrifuging, filtering, etc., may assist in controlling physical characteristics of the resulting tissue-derived matrix, e.g., whether a paste, putty, gel, or dispersion is formed and the rheological properties thereof, followed by formation of the matrix into a sheet or planar shape suitable to produce the sheet shaped body therefrom. In some embodiments, formation of the matrix into a suitable sheet or planar shape may include one or more further processing techniques such as, without limitation, lyophilizing, dehydrating, cross-linking, curing, and the like. Formation of the matrix into a suitable sheet or planar shape may or may not involve use of one or more devices such as a mold, sieve or filter.

According to some embodiments, pre-processing step (b) is performed at a temperature of about 25° C. According to some embodiments, pre-processing, step (b) is performed at a temperature of from about 4° C. to about 10° C., According to some embodiments, pre-processing step (b) is performed at an ambient temperature. According to some embodiments, pre-processing step (b) is performed at a temperature greater than an ambient temperature. According to some embodiments, pre-processing step (b) is performed at a physiological temperature of a living mammal. According to some embodiments, pre-processing step (b) is performed at a temperature of about 37° C.

The delipidizing step (c) is optional because, depending on the tissue type involved, delipidizing will not always be necessary or beneficial, as will be recognized and understood by persons of ordinary skill in the relevant art. For example, without limitation, where the tissue sample(s) consist of cartilage tissue, delipidizing would probably not be worthwhile. On the other hand, without limitation, where the tissue sample(s) comprise adipose tissue, delipidizing would probably be quite advantageous. Additionally, depending on the intended use of the volumizing graft, leaving lipid in the tissue sample(s), without delipidizing, may be preferred, as will be determinable by persons of ordinary skill in the relevant art. According to some embodiments, multiple delipidizing steps are performed. According to some embodiments, delipidizing step (c) is not performed.

According to some embodiments, delipidizing step (c) is a step of removing lipids from the tissue sample(s). Some disruption of cellular membranes and removal of cells may also occur. According to some embodiments, delipidizing step (c) comprises removing some of the lipids native to the tissue sample(s). According to some embodiments, delipidizing step (c) comprises removing most (i.e., at least 50% by weight) of the lipids native to the tissue sample(s). According to some embodiments, delipidizing step (c) comprises removing substantially all (i.e., at least 80% by weight, or at least 90% by weight, or at least 95% by weight) of the lipids native to the tissue sample(s). According to some embodiments, delipidizing step (c) removes substantially all of the lipids native to the tissue sample(s).

According to some embodiments, delipidizing step (c) disrupts cellular membranes of cells resident in the tissue sample(s). According to some embodiments, delipidizing step (c) removes lipids from the tissue sample(s). According to some embodiments, delipidizing step (e) is performed before the decellularizing step (d). According to some embodiments, delipidizing step (c) is performed after the decellularizing step (d).

According to some embodiments, the delipidizing step (c) comprises contacting the tissue sample(s) with a liquid (e.g., a delipidizing solution), with or without agitation, to separate lipids from the tissue sample(s). According to some embodiments, delipidizing step (e) comprises immersing, soaking, blending, homogenizing, etc., the tissue sample(s) in the liquid. According to some embodiments, blending includes a step of mixing the tissue sample(s) in the liquid under conditions of high shear. According to some embodiments, delipidizing step (c) comprises soaking the tissue sample(s) in the liquid, with or without agitation. According to some embodiments, homogenization includes a step of mixing the tissue sample(s) in the liquid such that the resulting homogenized tissue is evenly distributed throughout the liquid.

According to some embodiments, the liquid comprises water, According to some embodiments, the liquid comprises an organic solvent. According to some embodiments, the liquid is a mixture of organic solvents. According to some embodiments, the liquid is an aqueous mixture comprising one or more organic solvents and water. According to some embodiments, the organic solvent is selected from: a paraffin, an aromatic hydrocarbon, a cyclic hydrocarbon, a chlorinated hydrocarbon, a fluorinated hydrocarbon, a chlorinated methane, a fluorinated methane, an alcohol, an ether, a ketone, an organic acid, an aldehyde, an ester, and combinations thereof. According to some embodiments, the organic solvent may have one, two, three, four, five, or six carbon atoms, or combinations thereof when the liquid is a mixture of one or more organic solvents, with or without water. According to some embodiments, the liquid may include one or more of: an organic acid, a mineral acid, an organic base, a mineral base, an organic salt, and a mineral salt.

The particular techniques applied for delipidizing step (c), including the content of the delipidizing solution or liquid and the number of steps applied for a particular techniques or combination thereof, will depend upon the type of tissue sample being processed. According to some embodiments, such as those involving processing of adipose tissue sample(s), delipidizing step (c) may comprise contacting the tissue sample(s) with a liquid comprising a C₂ to C₅ carbon alcohol, such as, without limitation, 1-propanol. According to some embodiments, such as those involving processing of adipose tissue sample(s), delipidizing step (c) may comprise two or more blending steps, wherein the tissue sample(s) are blended in the liquid. In some embodiments, each blending step may be performed for a period of time which is from about 30 to about 120 seconds, wherein the periods of blending time need not be the same for each blending step. According to some embodiments, such as those involving processing of adipose tissue sample(s), delipidizing step (c) comprises two or more soaking steps, wherein the tissue sample(s) are soaked in the liquid. In some embodiments, each soaking step may be performed for a period of time which is from about 5 to about 24 hours, such as from about 5 minutes to about 18 hours, or from about 5 minutes to about 12 hours, or from about 5 minutes to about 6 hours, or from about 5 minutes to about 2 hours, or even from about 5 minutes to about 60 minutes, where the periods of soaking time need not be the same for each soaking step. In some embodiments, delipidizing step (c) comprises two blending steps with the liquid, each of which is performed for a blending period of about 60 to about 100 seconds, such as for about 80 seconds each, and further comprises two soaking steps with the liquid, each of which is performed for a soaking time of about 10 to about 30 minutes.

According to some embodiments, delipidizing step (c) comprises recovering a lipid layer, which may be a mixture of lipid and the aforesaid liquid, from the tissue sample(s). According to some embodiments, delipidizing step (c) may comprise recovering the lipid layer by one or more techniques selected from: differential settling, centrifugation, filtration, and decantation.

According to some embodiments, delipidizing step (c) comprises recovering the delipidized tissue. According to some embodiments, delipidizing step (c) may comprise recovering the delipidized tissue by one or more techniques selected from: differential settling, centrifugation, filtration, and decantation.

According to some embodiments, delipidizing step (e) comprises contacting the tissue sample(s) with a supercritical fluid (e.g., supercritical carbon dioxide). According to some embodiments, delipidizing step (c) comprises recovering the lipid by evaporation of the supercritical fluid.

According to some embodiments, delipidizing step (c) is performed at a temperature of from about 18° C. to about 25° C. According to some embodiments, delipidizing step (c) is performed at an ambient temperature. According to some embodiments, delipidizing step (c) is performed at a temperature greater than an ambient temperature. According to some embodiments, delipidizing step (c) is performed at a physiological temperature of a living mammal. According to some embodiments, delipidizing step (c) is performed at a temperature of from about about 35° C. to about 42° C., or any temperature therebetween, such as about 37° C., or about 40° C.

The decellularizing step (d) is optional because, depending on the tissue type involved, decellularizing will not always be necessary or beneficial, as will be recognized and understood by persons of ordinary skill in the relevant art. For example, without limitation, where the tissue sample(s) consist of amnion tissue, decellularizing may not be worthwhile given the perceived non-immunogenic nature of the cells found in amnion tissue. On the other hand, without limitation, where the tissue sample(s) comprise adipose tissue or dermal tissue, decellularizing would probably be quite advantageous. Additionally, depending on the intended use of the volumizing graft, leaving native cells in the tissue sample(s), without decellularizing, may be preferred, as will be determinable by persons of ordinary skill in the relevant art. According to some embodiments, multiple decellularizing steps may be performed. According to some embodiments, decellularizing step (c) is not performed. According to some embodiments, decellularizing step (d) is performed after a delipidizing step. According to some embodiments, decellularizing step (d) is performed before a delipidizing step,

According to some embodiments, decellularizing step (d) comprises a step of removing cells and cell fragments from tissue sample(s). According to some embodiments, decellularizing step (d) converts the tissue sample(s) to acellular matrix comprising primarily extracellular matrix (“ECM”). According to some embodiments, the acellular matrix is essentially free of cell fragments. According to some embodiments, the acellular matrix is entirely free of cell fragments. According to some embodiments, the acellular matrix is free of native tissuegenic factors. According to some embodiments, the acellular matrix includes native tissuegenic factors.

According to some embodiments, decellularizing step (d) comprises contacting the tissue sample(s) with a decellularizing solution. According to some embodiments, the tissue sample(s) is in contact with the decellularizing solution for at least about 6 hours, such as for at least about 8 hours, or at least about 10 hours, or at least about 12 hours, or even longer. According to some embodiments, decellularizing step (d) comprises contacting the tissue sample(s) with a decellularizing solution, with or without agitation, to disrupt the cells and remove cells and cell fragments from the tissue sample(s).

According to some embodiments, contacting the tissue sample(s) with a decellularizing solution in decellularizing step (d) may comprise immersing, soaking, agitating, blending, homogenizing, etc., the tissue sample(s) in the decellularizing solution. According to some embodiments, blending includes a step of mixing the soft tissue in the decellularizing solution under conditions of high shear. According to some embodiments, homogenizing includes a step of mixing the soft tissue in the decellularizing solution such that the tissue is evenly distributed throughout the liquid.

According to some embodiments, the decellularizing solution is hypertonic relative to the interior of the cells in the tissue sample(s). According to some embodiments, the decellularizing solution is hypotonic relative to the interior of the cells in the tissue sample(s). According to some embodiments, the decellularizing solution includes a salt and is a salt solution. According to some embodiments, the salt is sodium chloride. According to some embodiments, the decellularizing solution is a pH-buffered solution. According to some embodiments, the pH-buffered solution has a physiological pH. According to some embodiments, the pH-buffered solution has a pH of about 7.4.

According to some embodiments, the decellularizing solution includes a detergent, an emulsifier, a surfactant, or a combination thereof. According to some embodiments, at least one of the detergent, the emulsifier, and the surfactant includes one or more of: a derivative of a long chain fatty acid, sodium deoxycholate (SDC), sodium lauryl sulfate (SLS), sodium dodecyl sulfate (SDS), a non-ionic surfactant, a polyoxyethylene derivative of a long-chain fatty acid, a polyoxyethylene sorbitan monolaurate, polyethylene glycol or its derivatives (e.g., polyethylene glycol tert-octylphenyl ether, which is commercially available under the tradename Triton X-100® from Sigma Aldrich of St. Louis, Missouri, U.S.A.), and an aromatic hydrocarbon. The selection of particular the detergent, emulsifier, surfactant, and combinations thereof will depend upon the type of tissue sample being processed and it is within the ability of persons of ordinary skill in the relevant art to make such selections and combinations.

According to some embodiments, the detergent, emulsifier, surfactant, or combination thereof is present in a solvent at a concentration in the range of from about 0.1% to about 5.0% (w/v). According to some embodiments, the solvent includes water, According to some embodiments, the solvent includes an organic solvent. According to some embodiments, the solvent is an aqueous solvent which includes a mixture of water and an organic solvent. According to some embodiments, the solvent includes less than 20% organic solvent by volume. According to some embodiments, the solvent includes from about 20% about 40% organic solvent by volume. According to some embodiments, the solvent includes from about 40% to about 60% organic solvent by volume. According to some embodiments, the solvent includes from about 60% to about 80% organic solvent by volume. According to some embodiments, the solvent includes more than 80% organic solvent by volume.

According to some embodiments, the organic solvent is selected from a group consisting of a paraffin, an aromatic hydrocarbon, a cyclic hydrocarbon, a chlorinated hydrocarbon, a fluorinated hydrocarbon, a chlorinated methane, a fluorinated methane, an alcohol, an ether, a ketone, an aldehyde, an ester, an organic acid, and combinations thereof. According to some embodiments, the organic solvent may have one, two, three, four, five or six carbon atoms, or combinations thereof when the liquid is a mixture of one or more organic solvents, with or without water.

According to some embodiments, the decellularizing solution may include one or more of: an enzyme, a lipase, a collagenase, trypsin, an endonuclease, protease, and a protease inhibitor. According to some embodiments, decellularizing step (d) comprises a step of scraping a cellular layer from one or more of the tissue sample(s).

According to some embodiments, the decellularizing solution is mildly alkaline. According to some embodiments, the decellularizing solution is mildly acidic. In some embodiments, the decellularizing solution has a ph that is less than 6. According to some embodiments, the decellularizing solution has a pH in the range of from about 6 to about 8. In some embodiments, the decellularizing solution has a pH that is greater than 10. According to some embodiments, the decellularizing solution includes peracetic acid.

According to some embodiments, the tissue sample(s) are in contact with the decellularizing solution for at least 6 hours. According to some embodiments, the tissue sample(s) are in contact with the decellularizing solution for at least 12 hours. According to some embodiments, the tissue sample(s) are in contact with the decellularizing solution for at least 18 hours. According to some embodiments, the tissue sample(s) are in contact with the decellularizing solution for at least 24 hours. According to some embodiments, the tissue sample(s) are in contact with the decellularizing solution for at least 36 hours, According to some embodiments, the tissue sample(s) are in contact with the decellularizing solution for at least 48 hours. The contact time for the tissue sample and decellularizing solution (i.e., decellularizing time) is determinable by persons of ordinary skill in the relevant art based on, for example without limitation, the type of tissue being decellularized, the degree of decellularization desired and the content and concentration of the decellularizing solution.

For example without limitation, where the tissue sample(s) comprise dermal tissue, the tissue sample(s) may be in contact with a decellularizing solution comprising sodium chloride for a period of from about 12 to about 24 hours and, separately, with a decellularizing solution comprising Triton X-100 for a period from about 24 to about 48 hours. Where the tissue sample(s) comprise adipose tissue, fascial tissue, or both, the tissue sample(s) may be in contact with a decellularizing solution comprising sodium deoxycholate (SDC) for a period of from about 12 to about 24 hours.

According to some embodiments, decellularizing step (d) is performed at a temperature of about 25° C., According to some embodiments, decellularizing step (d) is performed at an ambient temperature. According to some embodiments, decellularizing step (d) is performed at a temperature greater than an ambient temperature. According to some embodiments, decellularizing step (d) is performed at a physiological temperature of a living mammal. According to some embodiments, decellularizing step (d) is performed at a temperature of about 37° C.

Blood and some blood components, as well as some cells and cell components, are generally considered to be immunogenic substances. It is advantageous and often preferred for grafts to be non-immunogenic and, therefore, removal of immunogenic substances from tissue sample(s) being formed into one or more grafts is generally believed to be advantageous, and sometimes necessary. Isolating step (a) often removes blood and blood components from tissue sample(s). Decellularizing step (d) removes cells and cell components from tissue sample(s). Accordingly, in embodiments where tissue sample(s) are processed by performing one or both of isolating step (a) and decellularizing step (d), the resulting tissue sample(s) and the sheet shaped body or additional components produced therefrom, will be at least partially free of immunogenic substances. In embodiments in which the volumizing grafts are produced using tissue sample(s) subjected to both isolating step (a) and decellularizing step (a) may be substantially free of immunogenic substances.

According to some embodiments, disinfecting step (e) comprises disrupting and/or removing micro-organisms, viruses, or both, from the tissue sample(s). According to some embodiments, disinfecting step (e) comprises contacting the tissue sample(s) with a disinfecting solution, with or without agitation. According to some embodiments, disinfecting step (e) comprises immersing, soaking, agitating, blending, etc., the tissue sample(s) in the disinfecting solution. According to some embodiments, blending includes a step of mixing the tissue sample(s) in the disinfecting solution under conditions of high shear.

According to some embodiments, the disinfecting solution may include one or more antibiotics. Antibiotics include, without limitation, amoxicillin, penicillin, gentamicin, amphotericin, doxycycline, azithromycin, vancomycin, and combinations thereof. According to some embodiments, the disinfecting solution includes an alcohol. According to some embodiments, the disinfecting solution includes a glycol. According to some embodiments the disinfecting solution includes a mixture of water with an alcohol, a glycol, or both. According to some embodiments, the disinfecting solution may include one or more of: a peroxy compound (e.g., peracetic acid), chlorine dioxide, a detergent, a surfactant, an ethylene diamine salt (e.g., ethylene diamine tetraacetic acid (EDTA)), a protein denaturant (e.g., a chaotropic salt, such as guanidine isothiocyanate, etc.).

In some embodiments, the disinfection solution includes one or more of: water (preferably deionized or distilled), ethanol, propylene glycol, and peracetic acid. In some embodiments, for example without limitation, the disinfection solution may include water mixed with 95% ethanol (aqueous), with or without one or both of peracetic acid and propylene glycol. In some embodiments, for example without limitation, the disinfection solution may include from about 0.05% to about 5%, by weight (w/w), of peracetic acid in water, or in a mixture of water and ethanol, with or without propylene glycol. For example, without limitation, peracetic acid may be present in the disinfection solution in a concentration of from about 0.5% to about 1%.

According to some embodiments, disinfecting step (e) is performed at a temperature of about 25° C. According to some embodiments, disinfecting step (e) is performed at an ambient temperature. According to some embodiments, disinfecting step (e) is performed at a temperature greater than an ambient temperature. According to some embodiments, disinfecting step (e) is performed at a physiological temperature of a living mammal. According to some embodiments, disinfecting step (e) is performed at a temperature of about 37° C.

According to some embodiments, disinfecting step (e) is performed at a pH in the range of from about 2 to about 8, such that the tissue sample(s) forms a flowable gel. According to some embodiments, disinfecting step (e) is performed at a pH in the range of about 4 to about 8 such that the tissue sample(s) forms a flowable gel. According to some embodiments, disinfecting step (e) is performed at a pH below the isoelectric point of collagen.

According to some embodiments, the disinfected tissue sample(s) is washed with water to remove the disinfecting solution from the disinfected tissue sample(s). According to some embodiments, the disinfected tissue sample(s) is washed with a buffer solution to remove the disinfecting solution from the disinfected tissue sample(s). According to some embodiments, the disinfected tissue sample(s) is washed with a physiological buffer to remove the disinfecting solution from the disinfected tissue sample(s) and bring the disinfected tissue to a physiological pH. According to some embodiments, the disinfected tissue sample(s) is washed with a solution containing a polar solvent to remove the disinfecting solution from the disinfected tissue sample(s),

According to some embodiments of the method for producing the sheet shaped body of the volumizing graft, post-processing step (f) comprises reshaping the at sheet shaped body (whether or not comprising a tissue derived matrix), which has a first surface and a second surface opposite the first surface, into the shape of a pouch, sling, balloon, wrap, cap, covering, container, retainer, envelope, or any other shape which includes the aforesaid cavity defined at least partially by the first surface of the body. The cavity receives and retains, for at least a period of time, therein one or more additional components.

In some embodiments, the method for producing the sheet shaped body of the volumizing graft may comprise attaching, arranging, or otherwise combining two or more sheets of tissue-derived matrix, each of which has a first surface and a second surface opposite the first surface thereof, with one another to form the retainer having the interior cavity. The step of reshaping of the at least one sheet of tissue-derived matrix may comprise using a container or mold to hold the at least one sheet into the desired configuration while a stabilizing step is performed, such as without limitation, dehydrating (e.g., air drying, heating, lyophilizing, etc.), cross-linking (e.g., physical, chemical, radiation exposure, etc.), and other means known and routinely used by persons of ordinary skill in the relevant art.

According to some embodiments, post-processing step (f) further comprises forming, or providing and attaching, one or more of the above-mentioned additional features to the sheet shaped body, such as the attachment features (e.g., tabs, straps, ribbons, or other extensions, one or more sutures already inserted through the body, or one or more apertures through the body for receiving sutures therethrough, etc.), pass-through features or expansion features (e.g., perforations, openings, slots, meshing, grooves, channels, etc.), and combinations thereof.

According to some embodiments, a method for producing a tissue derived matrix which is suitable for making the sheet shaped body of the volumizing grafts may further comprise a sterilizing step. Such a sterilizing step may be performed at any point or step during the production of the tissue derived graft and the sheet shaped body, including after all other steps are performed, and comprises exposing the one or more tissue samples to gamma radiation, electron beam radiation, ionizing radiation, chemical agents (e.g., alcohol, phenol, ethylene oxide gas, acids, bases, or peroxides), heat, or ultraviolet radiation for sufficient duration and dosages. In some embodiments, such a sterilizing step may comprise terminal sterilization which is generally performed on the finished volumizing graft, including the sheet shaped body and any other components, materials and substances that are assembled to form the graft, after placement in its final packaging.

The volumizing graft described and contemplated herein may further comprise an additional component, such as one, two, or more additional components, at least one of which is positioned and contained at least partially within the cavity of the sheet shaped body. Each additional component comprises one or more biocompatible materials, which may also be bioactive or not. Furthermore, each additional component may have or be in any physical form provided it is capable of being held and retained for at least a period of time in the cavity of the sheet shaped body, which will depend also, at least in part, on the configuration and material of the sheet shaped body, as will be described in further detail below. Furthermore, each of the additional components may, independently of the others, be assembled with the sheet shaped body by placing or positioning it into the cavity of the sheet shaped body either before, during, or after, the sheet shaped body is implanted at a treatment site of a subject. Each additional component may or may not be at least partially affixed, fastened, contained, or otherwise retained, in the cavity of the sheet shaped body using one or more devices such as, without limitation, fasteners, sutures, adhesives, lids, covers, snaps, ties, ribbons, and the like.

In some embodiments, a volumizing graft comprising a sheet shaped body which is either already in a shape having a cavity or capable of being reshaped into a shape having a cavity will be provided. The volumizing graft may further include one or more additional components and, in such embodiments, each of the one or more additional components may or may not, independently of the others, be assembled and positioned within the cavity of the sheet shape body. In further exemplary embodiments, one or more of the additional components may be at least partially positioned and contained in the cavity of the sheet shaped body.

In embodiments which include both the sheet shaped body and one or more additional components, but the sheet shaped body is provided in a form which needs to be reshaped to form a shape having the cavity, the sheet shaped body should first be reshaped to include the cavity, and then one or more of the additional components may be positioned in the cavity of the sheet shaped body so that it is at least partially covered and contained therein.

In any embodiment of the restraining graft which includes a sheet shaped body and one or more additional components which are not yet assembled with at least one of the additional components already positioned in the cavity, the graft may be assembled prior to implanting the volumizing graft at the treatment site. Alternatively, a sheet shaped body with a shape having the cavity may first be implanted at a treatment site, followed by placement, positioning, insertion, injection, or a combination of such techniques, of one or more additional components into the cavity of the body of the implanted graft.

The physical form and material of suitable additional components are not especially limited and are selected according to several factors including, without limitation, the intended use of the volumizing graft such as the surgical procedure to be performed and the condition being treated using the volumizing graft, as well as the location, condition and size of the treatment site, and the physical form (e.g., size and shape) of the sheet shaped body of the volumizing graft.

Each additional component may comprise any one or more natural or synthetic biocompatible materials. Natural biocompatible materials include, without limitation, tissue derived matrices, which include extracellular matrix biomaterials, such as collagen, proteins; natural-and animal-derived biomaterials, such as chitosan or alginate; and metals such as titanium, tantalum, etc. Synthetic biocompatible materials include, without limitation, ceramics, polymers (e.g., nylon, polyester, polypropylene, polyglycolic acid, etc.), resins, metal alloys (e.g., stainless steel, nickel-titanium, chromium-cobalt, etc.), and other materials (e.g., tri-calcium phosphate, hydroxyapatite, bioactive glass, etc.).

Generally, it is contemplated that the additional component may be in any physical form which is capable of being positioned and retained, at least partially, in the cavity of the sheet shaped body for at least some period of time. The at least some period of time for an additional component to be retained in the cavity of the sheet shaped body may be relatively short, such as from minutes or an hour, or longer such as up to several days, weeks, or months. In some embodiments, after implantation at a treatment site, at least additional component may be positioned and retained at least partially, or fully contained, in the cavity of the sheet shaped body of the graft, with the intention that the remain at the treatment site.

Physical forms which are suitable for the additional components of the volumizing grafts described and contemplated herein are not particularly limited and generally include, without limitation, (1) a pre-formed three dimensional shape which has been molded, manufactured, cut, machined, assembled, etc., and is capable of retaining its own shape in the absence of external support or containment; (2) a particulate form which may be at least partially dehydrated or not; (3) a flowable mixture, such as a liquid, a paste, a putty, or a gel. In some embodiments, the (2) particulate form may be combined with one or more biocompatible carriers to produce (3) a flowable mixture. Furthermore, as mentioned above, an additional component having the physical form of either (2) a particulate form or (3) a flowable mixture may be formulated, shaped and stabilized, such as by at least partially dehydrating or crosslinking, to produce an additional component having a physical form of (1) a pre-formed three dimensional shape.

Examples of additional components having a physical form which is (1) a pre-formed three dimensional shape include, without limitation, an implant, with or without additional features such as indents, cavities, channels, pockets, extensions, protrusions, surface texture, etc. Such implants include, without limitation, a breast implant (e.g., silicon-filled, saline-filled, etc.), a tissue extender, a gluteal implant, chin implant, calf implant, bicep implant, abdominal implant, etc. In such embodiments, the volumizing grafts may be useful to supplement or replace grafts or implants currently used for large, mid-, and small volume repair and reconstruction procedures. Other examples of additional components having a physical form which is (1) a pre-formed three dimensional shape include those which comprise a biocompatible material which is initially in the form of particulates, pieces, fibers, chunks, smaller components or shapes, a putty, or a paste, and has been subjected to shaping in a mold or by manual manipulation, with or without dehydrating, to provide the pre-formed three dimensional shape. In some embodiments, the biocompatible material is a tissue derived matrix.

According to some embodiments of the volumizing graft which further comprise an additional component having a physical form which is (1) a pre-formed three dimensional shape, the sheet shaped body of the volumizing graft may be shaped and applied to (e.g., wrapped around, layered upon, adhered to, or combinations thereof) around at least a portion of the pre-formed three dimensional shaped component so that it is at least partially covered and contained in a cavity which conforms to at least a portion of the pre-formed three dimensional shaped component. In some such embodiments, the sheet shaped body may completely cover and contain the pre-formed three dimensional shaped component. According to some such embodiments, the sheet shaped body is reshaped by folding or curling it to form a pouch or envelope having a cavity which receives at least a portion, or even all, of the implant therein.

According to some such embodiments, the sheet shaped body of the volumizing graft may comprise a tissue-derived matrix produced from one or more tissue samples, as described hereinabove. It is contemplated that the sheet shaped body may comprise two or more sheet shaped components, each of which comprises a tissue derived matrix. The use of two or more sheet shaped components to form the sheet shaped body may provide the ability to cover a greater portion, or even all, of a pre-formed three dimensional shape, or provide a thicker covering over the pre-formed three dimensional shape, than is otherwise possible using only one sheet shaped component.

In some embodiments of the volumizing graft in which the additional component is a pre-formed three dimensional shape, the sheet shaped body may be wrapped around and cover at least a portion of the implant of the pre-formed three dimensional shape and comprise at least one sheet shaped component, each of which comprises a tissue-derived matrix comprising one or more tissue types selected from: adipose tissue, fascia tissue, dermal tissue, muscle tissue, and combinations thereof. In such embodiments, the sheet shaped body of the volumizing graft may comprise two or more sheet shaped components which are layered with one another, each of which being derived from the same or different tissue types than the other layers. When such a volumizing graft is used for breast or gluteal (buttocks) reconstruction, augmentation, or both, the resulting reconstructed body feature may feel more natural. This is expected due to the potential for adipose cells to infiltrate and expand the matrix of the sheet shaped body and create a substantial thickness between the pre-formed three dimensional shape and the recipient's skin. In addition to improving the “feel” of a pre-formed three dimensional shape such as a breast implant, such a thickness could also potentially reduce the appearance of post-operative rippling or animation defect at the treatment site.

In some embodiments, a volumizing graft comprises a sheet shaped body and further comprises an additional component having a physical form which is either (2) a particulate form which may be partially dehydrated or not; or (3) a flowable mixture, such as a liquid, a paste, a putty, or a gel, which is not capable of maintaining its own shape in the absence of external support or containment. Generally, as contemplated herein, additional components having a physical form which is either (2) a particulate form or (3) a flowable mixture, are generally unable to maintain their own shape for any amount of time or, alternatively, they are able to maintain their own shape, in the absence of external support or containment (such as is provided by the sheet shaped body of the volumizing graft), for only a short time, such as less than about 24 hours, or less than about 18 hours, or less than about 12 hours, or less than about 6 hours, or less than about 4 hours, or less than about 2 hours, or less than about 1 hour, or less than about 30 minutes.

In such embodiments, the sheet shaped body and the one or more additional components having (2) particulate or (3) flowable mixture physical forms are each configured (e.g., sized and shaped) to enable the cavity of the sheet shaped body to receive and fully contain the additional component(s) therein. In other words, in these embodiments, the one or more additional components are completely surrounded and contained by the cavity of the sheet shaped body. In such embodiments of the volumizing graft, when the sheet shaped body is formed in a shape having a cavity, the sheet shaped body may be considered analogous to a balloon, pouch or envelope, in that may expand and further reshape as one or more additional components are provided to the cavity. In use, such embodiments of the volumizing graft may enable the positioning and implanting of the sheet shaped body at a treatment site prior to or during providing one or more additional components which are either (2) a particulate form or (3) a flowable mixture to the cavity. Alternatively, one or more such additional components (i.e., particulate or flowable mixture) may be assembled with the sheet shaped body by providing them to the cavity of the sheet shaped body prior to implanting the assembled volumizing graft at the treatment site. An additional component in the physical form of (3) a flowable mixture may be injectable or otherwise capable of delivery to and positioning in the cavity of the sheet shaped. body of a volumizing graft using a syringe, cannula, or tubing.

It is contemplated that in embodiments of the volumizing graft which further comprise an additional component having a physical form of either (2) a particulate form or (3) a flowable mixture, the additional component may, alternatively, be applied as a coating to at least a portion of a surface of the sheet shaped body (i.e., either the first surface which defines the cavity, or the second surface which faces the surrounding tissue of the treatment site). A particulate or flowable mixture additional component may be applied and adhered to at least a portion of a surface or surfaces of a sheet shaped body before, during, or after, the sheet shaped body is formed into a shape having a cavity (whether the sheet shaped body comprises one or more sheet shaped components of tissue-derived matrix or another biocompatible material) to form a coated volumizing graft.

For example, without limitation, an additional component having the physical form of (3) a flowable mixture comprising a tissue derived matrix may be applied (i.e., spread, layered, coated, distributed, etc.) onto at least a portion of a surface (e.g., the first surface, the second surface, or both) of the sheet shaped body of the volumizing graft, and then the volumizing graft may be subjected to dehydrating, such as by air drying, heating, evaporation, lyophilizing, etc., whereby the (3) flowable mixture of tissue derived matrix adheres to the sheet shaped body as a coating. Alternatively, in some embodiments of the method for producing the volumizing graft, a biocompatible adhesive such as, without limitation, fibrin glue, fibronectin, 1- and 2-component silicone adhesives, acrylic adhesives (including cyanoacrylates), epoxy-polyurethane blend adhesives, etc., may be used to adhere an additional component, whether comprising (2) a dehydrated or partially dehydrated particulate tissue-derived matrix, or (3) a flowable mixture comprising a particulate tissue-derived matrix, as a coating on at least a portion of a surface of the sheet shaped body. Furthermore, in some embodiments, one or more additional components may be affixed to the sheet shaped body of the volumizing graft by any effective and suitable bonding, attachment, or affixing techniques including, without limitation, crosslinking, covalent or ionic bonding, suturing, stapling, and the like.

In some embodiments, it is contemplated that a volumizing graft comprising a sheet shaped body which includes a tissue-derived matrix and which has been formed into a shape having a cavity, is provided to a user, and the user obtains, prepares, or both, an additional component comprising flowable material which includes autologous material such as, without limitation, lipoaspirate adipose tissue, or stromal vascular fraction, obtained from the intended recipient of the volumizing graft, at the time of the surgical procedure, which may for example, without limitation, be a fat grafting or similar reconstructive procedure. In either of such embodiments, the sheet shaped body of the graft may be implanted or positioned in or on the recipient and then filled with a quantity of one or more additional components, or the sheet shaped body may be first filled with a quantity of the one or more additional components and then implanted or positioned together in or proximate to a treatment site of recipient by the user (e.g., medical practitioner). In some embodiments, one or more additional components including, without limitation, autologous cells, fat graft, SVF, PRP, and the like, may be combined with the sheet shaped body of the volumizing graft at the time of use, i.e., before or during a procedure to implant the graft in a recipient.

According to some embodiments, the volumizing graft may comprise an additional component which comprises a tissue-derived matrix produced by processing one or more tissue samples recovered from one or more donors. It is contemplated that each tissue sample subject to such processing may, independently of other tissue samples, be autogenic, allogenic, xenogenic, or some combination thereof. Each of the one or more tissue samples which are suitable for processing to produce an additional component of the volumizing grafts described and contemplated herein may comprise one or more tissue types selected from: adipose tissue, an amnion tissue, an artery tissue, a bone tissue, a breast tissue a cartilage tissue, a connective tissue, a chorion tissue, a colon tissue, a non-calcified dental tissue, a dermal tissue, a duodenal tissue, an endothelial tissue, an epithelial tissue, a fascial tissue, a gastrointestinal tissue, a gingival tissue, a growth plate tissue, an intervertebral disc tissue, an intestinal mucosal tissue, an intestinal serosal tissue, a ligament tissue, a liver tissue, a lung tissue, a mammary tissue, a membranous tissue, a meniscal tissue, a muscle tissue, a nerve tissue, an ovarian tissue, a parenchymal organ tissue, a pericardial tissue, a periosteal tissue, a peritoneal tissue, a placental tissue, a skin tissue, a spleen tissue, a stomach tissue, a synovial tissue, a tendon tissue, a testes tissue, an umbilical cord tissue, a urological tissue, a vascular tissue, and a vein tissue. In some embodiments, the tissue sample comprises adipose tissue and, optionally, further comprises one or more tissue types selected from dermal tissue, fascial tissue, and muscle tissue. In some embodiments, each tissue sample comprises adipose and, optionally, one or more of dermal tissue, fascial tissue, and muscle tissue arranged in their original native configuration when recovered from one or more donors.

Tissue derived matrices may be produced in any of the physical forms described hereinabove in connection with the one or more additional components of the volumizing grafts described and contemplated herein. More specifically, as will be recognized and practicable by persons of ordinary skill in the relevant art, tissue derived matrices may be produced from one or more tissue samples using physical processes, chemical processes, or combinations thereof, to produce any of the following previously discussed physical forms: (1) a pre-formed three dimensional shape; (2) a particulate form; and (3) a flowable mixture, which have the characteristics discussed above. The (2) particulate form may be combined with a biocompatible carrier to form (3) a flowable mixture which may be a paste, a gel, a slurry, or a suspension, and at least some of which can be shaped and reshaped such as, without limitation, by manual manipulation, using a mold having a desired shape, using devices which cut, scrape, grate, etc., additive (3D) printing, or some combination of these techniques.

It is further contemplated that that embodiments of the volumizing graft further comprising an additional component with a physical form of either (1) a three dimensional shape or (3) a flowable mixture, may also be useful to supplement or replace a graft implanted to treat a body feature during autologous reconstruction procedures, where the graft would normally have been normally derived from tissue recovered from another part or feature of the recipient of the graft. In such cases, using the present volumizing graft could reduce or even eliminate the need to recover tissue from another part or feature of the recipient, or to create a donor flap, thereby reducing or eliminating secondary surgical sites and wounds for the recipient.

A tissue-derived matrix in particulate form may be derived from one or more tissue samples which comprise one or more tissue types listed above as suitable for the additional components and were recovered from a source comprising one or more donors. Methods for processing the one or more tissue samples to produce particulate tissue-derived matrices suitable to produce one or more additional components to be received and at least partially contained in the cavity of the sheet shaped body of the volumizing grafts include one or more of the following steps:
(a) isolating a sample of a soft tissue from its source;
(b) pre-processing each of the one or more tissue samples;
(c) optionally, delipidizing one or more of the one or more tissue samples;
(d) optionally, decellularizing one or more of the one or more tissue samples;
(e) optionally, disinfecting one or more of the one or more tissue samples; and
(f) post-processing the one or more tissue samples.

In various embodiments of the disclosed method, some of the aforesaid steps may be omitted, the order of the steps may be varied, or additional steps may be provided. The “one or more tissue samples” and the “tissue sample(s)” of the method may be the tissue sample(s) in the form isolated from the source, the pre-processed tissue sample(s), the delipidized tissue sample(s), the decellularized (or acellular) tissue sample(s), the disinfected tissue sample(s), the post-processed tissue sample(s), or the packaged tissue sample(s). Embodiments of the disclosed method are discussed further hereinbelow, and exemplary embodiments are presented.

According to some embodiments, isolating step (a) comprises excising the tissue sample(s) comprising desired tissue type(s) from its source. According to some embodiments. isolating step (a) comprises removing the tissue sample(s) comprising desired tissue type(s) from its source. According to some embodiments, isolating step (a) comprises aspirating the tissue sample(s) comprising desired tissue type(s) from its source. According to some embodiments, isolating step (a) comprises recovering the tissue sample(s) comprising desired tissue type(s) from its source. According to some embodiments, isolating step (a) comprises dissecting the tissue sample(s) comprising desired tissue type(s) from its source. In some embodiments, the source of the tissue sample(s) comprising desired tissue type(s) is a frozen source and the isolating step (a) includes thawing the source.

According to some embodiments, isolating step (a) comprises separating the tissue sample(s) which comprises the desired tissue type(s) from adjacent tissues of a different tissue type than the desired tissue type(s). According to some embodiments, isolating step (a) comprises cutting the adjacent tissues from the tissue sample(s). According to some embodiments, isolating step (a) comprises pulling the adjacent tissues away from the tissue sample(s). According to some embodiments, isolating step (a) comprises scraping the adjacent tissues from the tissue sample(s). According to some embodiments, isolating step (a) comprises separating the adjacent tissues from the tissue sample(s) by differential settling of the desired tissue type(s) and adjacent tissues in a liquid medium.

The source of the one or more tissue samples is the same as described and contemplated above for tissue samples recovered and processed to produce a sheet shaped body for volumizing grafts. Furthermore, each of the one or more tissue samples used to produce the particulate tissue-derived matrix may comprise one or more tissue types selected from the list provided above in connection with the additional components. In some embodiments, for example without limitation, at least one of the one or more tissue samples comprises an adipose tissue. In some embodiments, for example without limitation, at least one of the one or more tissue samples comprises an adipose tissue and further comprises one or more tissue types selected from dermal tissue, fascial tissue, and muscle tissue.

According to some embodiments, isolating step (a) is performed at a temperature of about 25° C. According to some embodiments, isolating step (a) is performed at a temperature of about 4° C. to about 10° C. According to some embodiments, isolating step (a) is performed at an ambient temperature.

According to some embodiments, isolating step (a) comprises reducing the bioburden of the tissue sample(s) before performing the isolating step (a). According to some embodiments, isolating step (a) comprises rinsing with a liquid prior to reduce bioburden levels on the surface of the tissue sample(s). According to some embodiments, the liquid comprises phosphate buffered saline (PBS). According to some embodiments, the liquid comprises acetic acid. According to some embodiments, the liquid comprises peracetic acid. According to some embodiments, the liquid comprises hydrogen peroxide. According to some embodiments, the bioburden of the tissue sample can be reduced by means of ionizing radiation.

According to some embodiments, pre-processing step (b) includes size reduction of the tissue sample(s), According to some embodiments, pre-processing step (b) comprises cutting the tissue sample(s) into strips. According to some embodiments, pre-processing step (b) comprises slicing the tissue sample(s). According to some embodiments, pre-processing step (b) comprises cutting the tissue sample(s) into chunks. According to some embodiments, pre-processing step (b) comprises mincing the tissue sample(s). According to some embodiments, pre-processing step (b) comprises grinding the tissue sample(s). According to some embodiments, pre-processing step (b) comprises milling the tissue sample(s). According to some embodiments, pre-processing step (b) comprises freezer milling the tissue sample(s). According to some embodiments, pre-processing step (b) comprises homogenizing the tissue sample(s).

According to some embodiments, pre-processing step (b) comprises separating components of the tissue sample(s) by differential settling of the components of the tissue sample(s). According to some embodiments, pre-processing step (b) comprises separating components of the tissue sample(s) by centrifuging. According to some embodiments, pre-processing step (b) comprises separating components of the tissue sample(s) by filtering. As will be recognized and understood by persons of ordinary skill in the relevant art, adjusting the parameters of any of the techniques used to perform pre-processing step (b), for example without limitation, grinding, milling, centrifuging, filtering, etc., may assist in controlling physical characteristics of the particulate tissue-derived matrix, e.g., whether a paste, putty, gel, or dispersion is formed and the rheological properties thereof.

According to some embodiments, pre-processing step (b) is performed at a temperature of about 25° C. According to some embodiments, pre-processing step (b) is performed at a temperature of from about 4° C. to about 10° C. According to some embodiments, pre-processing step (b) is performed at an ambient temperature. According to some embodiments, pre-processing step (b) is performed at a temperature greater than an ambient temperature. According to some embodiments, pre-processing step (b) is performed at a physiological temperature of a living mammal. According to some embodiments, pre-processing step (b) is performed at a temperature of about 37° C.

The delipidizing step (c) is optional because, depending on the tissue type involved, delipidizing will not always be necessary or beneficial, as will be recognized and understood by persons of ordinary skill in the relevant art. For example, without limitation, where the tissue sample(s) consist of cartilage tissue, delipidizing would probably not be worthwhile. On the other hand, without limitation, where the tissue sample(s) comprise adipose tissue, delipidizing would probably be quite advantageous. Additionally, depending on the intended use of the volumizing graft, leaving lipid in the tissue sample(s), without delipidizing, may be preferred, as will be determinable by persons of ordinary skill in the relevant art. According to some embodiments, multiple delipidizing steps are performed. According to some embodiments, delipidizing step (c) is not performed.

According to some embodiments, delipidizing step (c) is a step of removing lipids from the tissue sample(s). Some disruption of cellular membranes and removal of cells may also occur. According to some embodiments, delipidizing step (c) comprises removing some of the lipids native to the tissue sample(s). According to some embodiments, delipidizing step (c) comprises removing most (i.e., at least 50% by weight) of the lipids native to the tissue sample(s). According to some embodiments, delipidizing step (c) comprises removing substantially all (i.e., at least 80% by weight, or at least 90% by weight, or at least 95% by weight) of the lipids native to the tissue sample(s). According to some embodiments, delipidizing step (c) removes substantially all of the lipids native to the tissue sample(s).

According to some embodiments, delipidizing step (c) disrupts cellular membranes of cells resident in the tissue sample(s). According to some embodiments, delipidizing step (e) removes lipids from the tissue sample(s). According to some embodiments, delipidizing step (c) is performed before the decellularizing step (d). According to some embodiments, delipidizing step (c) is performed after the decellularizing step (d).

According to some embodiments, the delipidizing step (c) comprises contacting the tissue sample(s) with a liquid (e.g., a delipidizing solution), with or without agitation, to separate lipids from the tissue sample(s). According to some embodiments, delipidizing step (c) comprises immersing, soaking, blending, homogenizing, etc., the tissue sample(s) in the liquid. According to some embodiments, blending includes a step of mixing the tissue sample(s) in the liquid under conditions of high shear. According to some embodiments, delipidizing step (c) comprises soaking the tissue sample(s) in the liquid, with or without agitation. According to some embodiments, homogenization includes a step of mixing the tissue sample(s) in the liquid such that the resulting homogenized tissue is evenly distributed throughout the liquid.

According to some embodiments, the liquid comprises water. According to some embodiments, the liquid comprises an organic solvent. According to some embodiments, the liquid is a mixture of organic solvents. According to some embodiments, the liquid is an aqueous mixture comprising one or more organic solvents and water. According to some embodiments, the organic solvent is selected from: a paraffin, an aromatic hydrocarbon, a cyclic hydrocarbon, a chlorinated hydrocarbon, a fluorinated hydrocarbon, a chlorinated methane, a fluorinated methane, an alcohol, an ether, a ketone, an organic acid, an aldehyde, an ester, and combinations thereof. According to some embodiments, the organic solvent may have one, two, three, four, five, or six carbon atoms, or combinations thereof when the liquid is a mixture of one or more organic solvents, with or without water. According to some embodiments, the liquid may include one or more of: an organic acid, a mineral acid, an organic base, a mineral base, an organic salt, and a mineral salt.

The particular techniques applied for delipidizing step (c), including the content of the delipidizing solution or liquid and the number of steps applied for a particular techniques or combination thereof, will depend upon the type of tissue sample being processed. According to some embodiments, such as those involving processing of adipose tissue sample(s), delipidizing step (c) may comprise contacting the tissue sample(s) with a liquid comprising a C₂ to C₅ carbon alcohol, such as, without limitation, 1-propanol. According to some embodiments, such as those involving processing of adipose tissue sample(s), delipidizing step (c) may comprise two or more blending steps, wherein the tissue sample(s) are blended in the liquid. In some embodiments, each blending step may be performed for a period of time which is from about 30 to about 120 seconds, wherein the periods of blending time need not be the same for each blending step. According to some embodiments, such as those involving processing of adipose tissue sample(s), delipidizing step (c) comprises two or more soaking steps, wherein the tissue sample(s) are soaked in the liquid. In some embodiments, each soaking step may be performed for a period of time which is from about 5 to about 24 hours, such as from about 5 minutes to about 18 hours, or from about 5 minutes to about 12 hours, or from about 5 minutes to about 6 hours, or from about 5 minutes to about 2 hours, or even from about 5 minutes to about 60 minutes, where the periods of soaking time need not be the same for each soaking step. In some embodiments, delipidizing step (c) comprises two blending steps with the liquid, each of which is performed for a blending period of about 60 to about 100 seconds, such as for about 80 seconds each, and further comprises two soaking steps with the liquid, each of which is performed for a soaking time of about 10 to about 30 minutes.

According to some embodiments, delipidizing step (c) comprises recovering a lipid layer, which may be a mixture of lipid and the aforesaid liquid, from the tissue sample(s). According to some embodiments, delipidizing step (e) may comprise recovering the lipid layer by one or more techniques selected from: differential settling, centrifugation, filtration, and decantation.

According to some embodiments, delipidizing step (c) comprises recovering the delipidized tissue. According to some embodiments, delipidizing step (c) may comprise recovering the delipidized tissue by one or more techniques selected from: differential settling, centrifugation, filtration, and decantation.

According to some embodiments, delipidizing step (c) comprises contacting the tissue sample(s) with a supercritical fluid (e.g., supercritical carbon dioxide). According to some embodiments, delipidizing step (e) comprises recovering the lipid by evaporation of the supercritical fluid.

According to some embodiments, delipidizing step (c) is performed at a temperature of from about 18° C. to about 25° C. According to some embodiments, delipidizing step (c) is performed at an ambient temperature. According to some embodiments, delipidizing step (c) is performed at a temperature greater than an ambient temperature. According to some embodiments, delipidizing step (c) is performed at a physiological temperature of a living mammal. According to some embodiments, delipidizing step (c) is performed at a temperature of from about about 35° C. to about 42° C., or any temperature therebetween, such as about 37° C., or about 40° C.

The decellularizing step (d) is optional because, depending on the tissue type involved, decellularizing will not always be necessary or beneficial, as will be recognized and understood by persons of ordinary skill in the relevant art. For example, without limitation, where the tissue sample(s) consist of amnion tissue, decellularizing may not be worthwhile given the small quantity of cells expected to be found in amnion tissue. On the other hand, without limitation, where the tissue sample(s) comprise adipose tissue or dermal tissue, decellularizing would probably be quite advantageous. Additionally, depending on the intended use of the volumizing graft, leaving native cells in the tissue sample(s), without decellularizing, may be preferred, as will be determinable by persons of ordinary skill in the relevant art, According to some embodiments, multiple decellularizing steps may be performed. According to some embodiments, decellularizing step (e) is not performed. According to some embodiments, decellularizing step (d) is performed after a delipidizing step. According to some embodiments, decellularizing step (d) is performed before a delipidizing step.

According to some embodiments, decellularizing step (d) comprises a step of removing cells and cell fragments from tissue sample(s). According to some embodiments, decellularizing step (d) converts the tissue sample(s) to acellular matrix comprising primarily extracellular matrix (“ECM”). According to some embodiments, the acellular matrix is essentially free of cell fragments. According to some embodiments, the acellular matrix is entirely free of cell fragments. According to some embodiments, the acellular matrix is free of native tissuegenic factors. According to some embodiments, the acellular matrix includes native tissuegenic factors.

According to some embodiments, decellularizing step (d) comprises contacting the tissue sample(s) with a decellularizing solution. According to some embodiments, the tissue sample(s) is in contact with the decellularizing solution for at least about 6 hours, such as for at least about 8 hours, or at least about 10 hours, or at least about 12 hours, or even longer. According to some embodiments, decellularizing step (d) comprises contacting the tissue sample(s) with a decellularizing solution, with or without agitation, to disrupt the cells and remove cells and cell fragments from the tissue sample(s).

According to some embodiments, contacting the tissue sample(s) with a decellularizing solution in decellularizing step (d) may comprise immersing, soaking, agitating, blending, homogenizing, etc., the tissue sample(s) in the decellularizing solution. According to some embodiments, blending includes a step of mixing the soft tissue in the decellularizing solution under conditions of high shear. According to some embodiments, homogenizing includes a step of mixing the soft tissue in the decellularizing solution such that the tissue is evenly distributed throughout the liquid.

According to some embodiments, the decellularizing solution is hypertonic relative to the interior of the cells in the tissue sample(s). According to some embodiments, the decellularizing solution is hypotonic relative to the interior of the cells in the tissue sample(s). According to some embodiments, the decellularizing solution includes a salt and is a salt solution. According to some embodiments, the salt is sodium chloride. According to some embodiments, the decellularizing solution is a pH-buffered solution. According to some embodiments, the pH-buffered solution has a physiological pH. According to some embodiments, the pH-buffered solution has a pH of about 7.4.

According to some embodiments, the decellularizing solution includes a detergent, an emulsifier, a surfactant, or a combination thereof. According to some embodiments, at least one of the detergent, the emulsifier, and the surfactant includes one or more of: a derivative of a long chain fatty acid, sodium deoxycholate (SDC), sodium lauryl sulfate (SLS), sodium dodecyl sulfate (SDS), a non-ionic surfactant, a polyoxyethylene derivative of a long-chain fatty acid, a polyoxyethylene sorbitan monolaurate, polyethylene glycol or its derivatives (e.g., polyethylene glycol tert-octylphenyl ether, which is commercially available under the tradename Triton X-100® from Sigma Aldrich of St. Louis, Missouri, U.S.A.), and an aromatic hydrocarbon. The selection of particular the detergent, emulsifier, surfactant, and combinations thereof will depend upon the type of tissue sample being processed and it is within the ability of persons of ordinary skill in the relevant art to make such selections and combinations.

According to some embodiments, the detergent, emulsifier, surfactant, or combination thereof is present in a solvent at a concentration in the range of from about 0.1% to about 5.0% (w/v). According to some embodiments, the solvent includes water. According to some embodiments, the solvent includes an organic solvent. According to some embodiments, the solvent is an aqueous solvent which includes a mixture of water and an organic solvent. According to some embodiments, the solvent includes less than 20% organic solvent by volume. According to some embodiments, the solvent includes from about 20% about 40% organic solvent by volume. According to some embodiments, the solvent includes from about 40% to about 60% organic solvent by volume. According to some embodiments, the solvent includes from about 60% to about 80% organic solvent by volume. According to some embodiments, the solvent includes more than 80% organic solvent by volume.

According to some embodiments, the organic solvent is selected from a group consisting of a paraffin, an aromatic hydrocarbon, a cyclic hydrocarbon, a chlorinated hydrocarbon, a fluorinated hydrocarbon, a chlorinated methane, a fluorinated methane, an alcohol, an ether, a ketone, an aldehyde, an ester, an organic acid, and combinations thereof. According to some embodiments, the organic solvent may have one, two, three, four, five or six carbon atoms, or combinations thereof when the liquid is a mixture of one or more organic solvents, with or without water.

According to some embodiments, the decellularizing solution may include one or more of: an enzyme, a lipase, a collagenase, trypsin, an endonuclease, protease, and a protease inhibitor. According to some embodiments, decellularizing step (d) comprises a step of scraping a cellular layer from one or more of the tissue sample(s).

According to some embodiments, the decellularizing solution is mildly alkaline. According to some embodiments, the decellularizing solution is mildly acidic. In some embodiments, the decellularizing solution has a pH that is less than 6, According to some embodiments, the decellularizing solution has a pH in the range of from about 6 to about 8. In some embodiments, the decellularizing solution has a pH that is greater than 10. According to some embodiments, the decellularizing solution includes peracetic acid.

According to some embodiments, the tissue sample(s) are in contact with the decellularizing solution for at least 6 hours. According to some embodiments, the tissue sample(s) are in contact with the decellularizing solution for at least 12 hours. According to some embodiments, the tissue sample(s) are in contact with the decellularizing solution for at least 18 hours. According to some embodiments, the tissue sample(s) are in contact with the decellularizing solution for at least 24 hours. According to some embodiments, the tissue sample(s) are in contact with the decellularizing solution for at least 36 hours. According to some embodiments, the tissue sample(s) are in contact with the decellularizing solution for at least 48 hours.

For example without limitation, where the tissue sample(s) comprise dermal tissue, the tissue sample(s) may be in contact with a decellularizing solution comprising sodium chloride for a period of from about 12 to about 24 hours and, separately, with a decellularizing solution comprising Triton X-100 for a period from about 24 to about 48 hours. Where the tissue sample(s) comprise adipose tissue, fascial tissue, or both, the tissue sample(s) may be in contact with a decellularizing solution comprising sodium deoxycholate (SDC) for a period of from about 12 to about 24 hours.

According to some embodiments, decellularizing step (d) is performed at a temperature of about 25° C. According to some embodiments, decellularizing step (d) is performed at an ambient temperature. According to some embodiments, decellularizing step (d) is performed at a temperature greater than an ambient temperature. According to some embodiments, decellularizing step (d) is performed at a physiological temperature of a living mammal. According to some embodiments, decellularizing step (d) is performed at a temperature of about 37° C.

Blood and some blood components, as well as some cells and cell components, are generally considered to be immunogenic substances. It is advantageous and often preferred for gratis to be non-immunogenic and, therefore, removal of immunogenic substances from tissue sample(s) being formed into one or more grafts is generally believed to be advantageous, and sometimes necessary. Isolating step (a) often removes blood and blood components from tissue sample(s). Decellularizing step (d) removes cells and cell components from tissue sample(s). Accordingly, in embodiments where tissue sample(s) are processed by performing one or both of isolating step (a) and decellularizing step (d), the resulting tissue sample(s) and the additional components produced therefrom, will be at least partially free of immunogenic substances. In embodiments in which the volumizing grafts are produced using tissue sample(s) subjected to both isolating step (a) and decellularizing step (d) may be substantially free of immunogenic substances.

According to some embodiments, disinfecting step (e) comprises disrupting and/or removing micro-organisms, viruses, or both, from the tissue sample(s). According to some embodiments, disinfecting step (e) comprises contacting the tissue sample(s) with a disinfecting solution, with or without agitation. According to some embodiments, disinfecting step (c) comprises immersing, soaking, agitating, blending, etc., the tissue sample(s) in the disinfecting solution. According to some embodiments, blending includes a step of mixing the tissue sample(s) in the disinfecting solution under conditions of high shear.

According to some embodiments, the disinfecting solution may include one or more antibiotics. Antibiotics include, without limitation, amoxicillin, penicillin, gentamicin, amphotericin, doxycycline, azithromycin, vancomycin, and combinations thereof. According to some embodiments, the disinfecting solution includes an alcohol. According to some embodiments, the disinfecting solution includes a glycol. According to some embodiments the disinfecting solution includes a mixture of water with an alcohol, a glycol, or both. According to some embodiments, the disinfecting solution may include one or more of: a peroxy compound (e.g., peracetic acid), chlorine dioxide, a detergent, a surfactant, an ethylene diamine salt (e.g., ethylene diamine tetraacetic acid (EDTA)), a protein denaturant (e.g., a chaotropic salt, such as guanidine isothiocyanate, etc.).

In some embodiments, the disinfection solution includes one or more of: water (preferably deionized or distilled), ethanol, propylene glycol, and peracetic acid. In some embodiments, for example without limitation, the disinfection solution may include water mixed with 95% ethanol (aqueous), with or without one or both of peracetic acid and propylene glycol. In some embodiments, for example without limitation, the disinfection solution may include from about 0.05% to about 5%, by weight, of peracetic acid in water, or in a mixture of water and ethanol, with or without propylene glycol. For example, without limitation, peracetic acid may be present in the disinfection solution in a concentration of from about 0.5% to about 1%.

According to some embodiments, disinfecting step (c) is performed at a temperature of about 25° C. According to some embodiments, disinfecting step (e) is performed at an ambient temperature. According to some embodiments, disinfecting step (c) is performed at a temperature greater than an ambient temperature. According to some embodiments, disinfecting step (e) is performed at a physiological temperature of a living mammal. According to some embodiments, disinfecting step (e) is performed at a temperature of about 37° C.

According to some embodiments, disinfecting step (e) is performed at a pH in the range of from about 2 to about 8, such that the tissue sample(s) forms a flowable gel. According to some embodiments, disinfecting step (e) is performed at a pH in the range of about 4 to about 8 such that the tissue sample(s) forms a flowable gel. According to some embodiments, disinfecting step (e) is performed at a pH below the isoelectric point of collagen.

According to some embodiments, the disinfected tissue sample(s) is washed with water to remove the disinfecting solution from the disinfected tissue sample(s). According to some embodiments, the disinfected tissue sample(s) is washed with a buffer solution to remove the disinfecting solution from the disinfected tissue sample(s). According to some embodiments, the disinfected tissue sample(s) is washed with a physiological buffer to remove the disinfecting solution from the disinfected tissue sample(s) and bring the disinfected tissue to a physiological pH. According to some embodiments, the disinfected tissue sample(s) is washed with a solution containing a polar solvent to remove the disinfecting solution from the disinfected tissue sample(s).

According to some embodiments, post-processing step (f), as applied to tissue derived matrices being processed to produce an additional component to be received in the cavity of the sheet shaped body of a volumizing graft, comprises forming the processed tissue sample(s) into (2) a particulate form of tissue-derived matrix and then into a final physical form, whether that is (1) the pre-formed three dimensional shape such as may be formed from a particulate tissue-derived matrix or (3) the flowable mixture comprising the particulate tissue-derived matrix and a biocompatible carrier.

As contemplated herein, (2) a particulate tissue-derived matrix may be further processed to produce an additional component having the physical form of (1) a pre-formed three dimensional shape, which may have been at least partially dehydrated, and which retains its three dimensional shape until reshaped by a user (e.g., a medical practitioner) either manually or otherwise. Such reshaping may involve adding or combining one or more biocompatible carriers or liquids with (1) the pre-formed three dimensional additional component to form a flowable paste, putty, or other malleable, moldable mass. It is further contemplated herein, that the additional component may be provided as a “hydrated” (3) flowable mixture, which means that the (3) flowable mixture was not dehydrated, or was only partially dehydrated, so that it is already flowable and deliverable using a cannula, syringe or similar device. In some embodiments of the (3) flowable mixture form of an additional component, rehydrating with one or more biocompatible carriers (such as by a user, medical practitioner, etc.) is possible and may be advantageous to achieve the degree of flowability and viscosity desired and required to function with the particular delivery devices or uses intended.

According to some embodiments, post-processing step (f) includes size reduction of the processed tissue sample(s) to produce the particulate tissue-derived matrix, such as, without limitation, by milling, freezer-milling (i.e., milling the tissue sample(s) while in a frozen state, for example, by impact milling), grinding, shaving, grating, freeze-fracturing, etc., the processed tissue sample(s).

The average size of the particles or pieces of processed tissue sample should be selected based on the desired form of the additional component. For example, where the additional component is intended to be a powder which is relatively more fluid, less viscous, and able to form a slurry or fine dispersion or suspension, the size reduction should be performed to produce a particulate tissue derived matrix comprising a plurality of particles having an average particle size of from about 0.001 mm to about 2 mm. Alternatively, where the additional component is intended to be a more granular but still flowable and reshapable putty or paste which is more viscous that the aforesaid powder form and may even hold its shape somewhat after reshaping, the size reduction should probably not include grinding or milling, but rather should be performed by techniques which produce a particulate tissue-derived matrix comprising a plurality of larger particles having an average particle size of from about 5 mm to about 10 mm. In still another alternative embodiment, where the additional component is intended to be highly granular putty or mass, with pieces or chunks of tissue-derived matrix and having a flowability or viscosity which allows the particulate tissue-derived matrix to hold its shape even without dehydration, or with only partial dehydration, the size reduction should be performed by techniques which produce a particulate tissue-derived matrix comprising a plurality of pieces or chunks having an average particle size of from about 10 mm to about 10 cm.

Where it is desired to produce an additional component comprising (1) a pre-formed three dimensional shape formed from the particulate tissue-derived matrix, as will be recognized by persons of ordinary skill in the relevant art, in some embodiments, the particulate tissue-derived matrix may be placed in a container or mold and subjected to dehydrating, such as by air drying, heat drying, lyophilizing, etc., while in the container or mold. The dehydrating may produce an at least partially dehydrated three dimensional shape which is the same or approximate shape as that portion of the container or mold filled with the particulate tissue-derived matrix prior to dehydration.

An additional component comprising (1) a pre-formed three dimensional shape formed from the particulate tissue-derived matrix and which has been at least partially dehydrated will have from about 0.5% to about 15%, including any weight value therebetween, by weight (wt %) of water based on the total weight of the additional component comprising an at least partially dehydrated three dimensional shape. In some embodiments, the additional component comprising an at least partially dehydrated three dimensional shape has no more than about 15% wt % of water such as, for example without limitation, no more than about 12 wt %, or no more than about 10 wt %, or no more than about 8 wt %, or no more than about 6 wt %, or no more than about 4% wt %, or no more than about 2 wt %, or no more than about 1 wt %, of water.

Optionally, the particulate tissue-derived matrix may be combined or mixed with a biocompatible carrier prior to being placed in a container or mold and at least partially dehydrated. In some embodiments where the particulate tissue-derived matrix, whether combined or mixed with a biocompatible carrier or not, is manually shapable and capable of maintaining its shape while subjected to dehydrating, the particulate derived-matrix (with or without biocompatible carrier) may be manually manipulated and shaped into a desired three dimensional shape, and then subjected to dehydration. Where the particulate tissue-derived matrix itself is not cohesive and manually shapable, combining or mixing it with one or more biocompatible carriers may produce a paste, putty, or gel which is manually shapable and sufficiently cohesive to maintain the desired three dimensional shape during partial or complete dehydrating. The dehydration techniques are not particularly limited and it is well within the ability of persons of ordinary skill in the relevant art to select suitable dehydrating methods and conditions based on consideration of the amount, dimensions, and consistency of the particulate tissue-derived matrix.

According to some embodiments, a additional component comprising (2) a particulate tissue-derived matrix may be produced in the desired size and shape by performing an additive (three dimensional, “3D”) printing technique. As will be recognized by persons of ordinary skill in the relevant art, to employ a 3D printing technique, the particulate tissue-derived matrix would have been subjected to a post processing (f) step comprising size reduction to a particle size compatible with the 3D printing device(s) to be used. Additionally, the particulate tissue-derived matrix the post-processing (f) step may further comprise combining or mixing the particulate tissue-derived matrix with one or more biocompatible carriers to produce a particulate tissue-derived derived matrix having flowability and viscosity characteristics compatible with the 3D printing device(s) to be used.

According to some embodiments, post-processing step (f) comprises reshaping the (1) three dimensional shape formed from the particulate tissue-derived matrix by cutting, shaving, machining, heating, melting, chipping, grating, etc. to produce a different three dimensional shape.

Where the additional component of the volumizing graft is (1) a pre-formed three dimensional shape formed from the particulate tissue-derived matrix, the three dimensional shape may be any desired shape, including being regular or irregular, symmetrical or asymmetrical, a recognized geometrical shape or combination thereof, or not. Moreover, the (1) pre-formed three dimensional shape may be porous or not. It is believed that it would be particularly advantageous for (1) the pre-formed three dimensional shape produced from (2) a particulate tissue-derived matrix to be porous to allow for fluid passage and egress through and out of the additional component of the volumizing graft, as well as ingress and ingrowth of cells and new tissue into the additional component which is at least partially contained in the cavity of the sheet shaped body of the volumizing graft. The (1) pre-formed three dimensional shape of the additional component may be fibrous or not, independently of other features and characteristics. The (1) pre-formed three dimensional shape of the additional component may have spongy characteristics including, for example without limitation, the ability to readily and quickly absorb fluids while substantially maintaining the three dimensional shape.

According to some embodiments, post-processing step (f) of the method for producing an additional component may comprise perforating (1) a pre-formed three dimensional shape formed from a particulate tissue-derived matrix to provide one or more recesses, pores, passages, pathways, channels, or combinations thereof, on or through the three dimensional shape. Such recesses, pores, passages, pathways, and channels would allow for fluid passage and egress through and out of the additional component of the volumizing graft, as well as ingress and ingrowth of cells and new tissue into the additional component of the volumizing graft. Providing one or more recesses, pores, passages, pathways, channels, or combinations thereof in the additional component may encourage early cellular infiltration into the volumizing graft in embodiments in which the sheet shaped body at least partially surrounds and contains one or more additional components. In some embodiments wherein a particulate tissue-derived matrix is shaped in a container or mold to produce a pre-formed three dimensional shaped additional component, pores, passages, recesses, pores, passages, channels, or combinations thereof, may be formed as part of this molding process, for example, by using rods, protrusions or other features or means in the container or mold for creating such features.

As described earlier, in some embodiments, the additional component of the volumizing graft may comprise (3) a flowable mixture of (2) a particulate tissue-derived matrix and a biocompatible carrier. To produce the (3) flowable mixture suitable to be provided to and contained in the cavity of a sheet shaped body to form an assembled volumizing graft, the above-described method for producing the particulate tissue-derived matrix may be performed, including one or more of steps (a) through (e) as desired to isolate and process one or more tissue samples comprising at least one desired tissue type. In such embodiments, before performing any post-processing steps (f) which involve dehydrating, the resulting particulate tissue-derived matrix may already comprise (2) a flowable mixture wherein the biocompatible carrier comprises water not yet removed from the matrix. Such a flowable particulate tissue-derived matrix may suitably be used as an additional component to be provided to and contained in the cavity of the sheet shaped body.

In some embodiments, the particulate tissue-derived matrix produced by performing one or more of steps (a) through (e) may not be flowable, or not be sufficiently flowable to produce a volumizing graft having the desired combination of properties. In such circumstances, post-processing (f) may comprise combining and mixing (2) the particulate tissue-derived matrix with one or more biocompatible carriers to produce (3) a flowable mixture which may have the consistency of and be a paste, a putty, a gel, a slurry, or a dispersion. The resulting (3) flowable mixture may be used as an additional component to be contained in a cavity of the volumizing graft.

According to some embodiments, methods for producing (2) a particulate tissue-derived matrix which is useful as an additional component of the volumizing graft may further comprise a sterilizing step. Such a sterilizing step may be performed at any point or step during the production of the additional component, including after all other steps are performed, and comprises exposing the one or more tissue samples, the particulate tissue-derived matrix, or the additional component to gamma radiation, electron beam radiation, ionizing radiation, chemical agents (e.g., alcohol, phenol, ethylene oxide gas, acids, bases, or peroxides), heat, or ultraviolet radiation for sufficient duration and dosages. In some embodiments, such a sterilizing step may comprise terminal sterilization which is generally performed on the assembled volumizing graft in its final packaging.

It is contemplated, without limitation, that embodiments of the volumizing grafts which further comprise an additional component in the form of (1) an at least partially dehydrated pre-formed three dimensional shape and a sheet shaped body comprising at least one sheet of biocompatible material (whether tissue-derived matrix or other biocompatible material) that at least partially surrounds and contains (1) the pre-formed three dimensional shaped additional component, would be particularly suitable for procedures intended to reconstruct and reshape more static, less fluid, body features such as, without limitation, a chin or abdominal muscles.

It is contemplated, without limitation, that embodiments of the volumizing graft described hereinabove as further comprising an additional component comprising particulate tissue-derived matrix in the form of (2) a flowable mixture and the sheet shaped body comprises at least one sheet of biocompatible material (whether tissue-derived matrix or other biocompatible material) which surrounds and contains the additional component, would be particularly useful for procedures intended to provide a more fluid or physically adaptable reconstruction, such as in breast reconstruction procedures.

It is contemplated that some embodiments of the volumizing graft described hereinabove, especially those further comprising an additional component comprising a particulate tissue-derived matrix in the form of (2) a flowable mixture and the sheet shaped body comprises at least one sheet of biocompatible material (whether tissue-derived matrix or other biocompatible material) that surrounds and contains the additional component, would be suitable for use as substitutes in place of some implants, such as breast implants and gluteal implants used for reconstruction of a breast or gluteal body feature.

As mentioned above, the volumizing grafts described and contemplated hereinabove could potentially eliminate or reduce the need for a donor site normally created during autologous reconstruction procedures. Removing the need for a donor site, would reduce the number of wound sites on the patient (graft recipient), decrease time spent in the operating room, and potentially lead to quicker recovery times for patients (recipients) after a reconstruction or augmentation procedure.

According to some embodiments, the method for producing a volumizing comprising a sheet shaped body and further comprising an additional component, may further comprise a packaging step (g) which comprises producing the sheet shaped body and the additional component, preferably but not necessarily assembled or combined with one another into the volumizing graft, for one or more of storage, transport and subsequent use. According to some embodiments, packaging step (g) comprises immersing one or both of the sheet shaped body and additional component in a preservative solution. According to some such embodiments, the preservative solution comprises aqueous ethanol. According to some such embodiments, packaging step (g) comprises freezing one or both of the sheet shaped body and additional component, for one or more of storage and transport. According to some embodiments, packaging step (g) comprises packaging the volumizing graft in a dehydrated or lyophilized state for one or more of storage, transport and subsequent use. According to some embodiments, packaging step (g) comprises storing the dried or lyophilized volumizing graft at temperatures between 4° C. and an ambient temperature.

According to some embodiments, one or both of the sheet shaped body and additional component are packaged while in a frozen state. According to some embodiments, one or both of the sheet shaped body and additional component are provided in a frozen state, and then thawed before packaging. According to some embodiments, one or both of the sheet shaped body and additional component are not in a frozen state when provided, and are packaged while in the state in which they are provided.

According to some embodiments, one or both of the sheet shaped body and additional component of a volumizing graft may be subjected to a sterilizing step. More particularly, in some embodiments, the method for producing a particulate tissue-derived matrix useful to produce the additional component of the volumizing graft may further comprise a sterilizing step. Such a sterilizing step may be performed at any point or step during the production of the additional component, including after all other steps are performed and includes exposing the one or more tissue samples or additional component to ionizing radiation.

The volumizing grafts described and contemplated herein may provide grafts having improved mechanical strength for resisting or preventing suture tear-out, while also having sufficient flexibility to reshape and substantially conform to the body feature being treated and sometimes also to an implant positioned proximate the graft, or even being supported in whole or in part by the graft. The volumizing grafts described and contemplated herein may provide grafts having improved mechanical strength while also having elasticity sufficient to maintain some minimum size, surface area, or volume and not be reshaped or stretched to the point of failing to provide the necessary and desired degree of support and structure to the body feature.

Sometimes only a portion of a graft implanted to treat a body feature remains after being implanted at a treatment site long enough for tissue and cell ingrowth to restore mass and volume to the body feature being treated to the degree intended. In such cases, the volumizing grafts described and contemplated herein may remain and resist degradation or absorption at the treatment site for a longer period of time after implanting and, thereby, provide improved volume reconstruction and retention at the treatment site.

The volumizing grafts described and contemplated herein will provide an immediate volume filling effect at the treatment site, and may further provide volume expansion over time as new cells infiltrate tissue-derived matrices of the sheet shaped body, one or more additional components, or a combination thereof. Further volume expansion at a treatment site may be achieved, after the initial surgical reconstruction procedure using a volumizing graft as described and contemplated herein, by periodically and serially providing supplemental additional components comprising biocompatible material, which may or may not comprise tissue derived matrices, to the cavity of the sheet shaped body of the graft. The ability to revisit a treatment site at which a volumizing graft was previously implanted to inject or otherwise deliver or administer subsequent additional components to the volumizing graft (e.g., proximate to or in the cavity of the sheet shaped body of the implanted graft) may offer opportunities to more effectively treat post-operative infection or perform corrective or revisionist treatment which may or may not be necessitated due to post-operative shifting or other movement of implanted materials. This ability to revisit a treatment site may also offer opportunities to adjust the volumizing graft for potential volume loss over time.

Furthermore, it is contemplated that a volumizing graft implanted at a treatment site may provide volume and, thereafter, an additional component comprising an adipose tissue derived matrix (which may be autologous fat or lipoaspirate, but does not have to be) may be injected into new void space created by the aforesaid volume. This approach and others mentioned herein may be useful during performance of breast lumpectomies and subsequent repair and reconstruction of the resulting void area, for example by producing and implanting a volumizing graft comprising a sheet shaped body comprising a dermal tissue derived matrix and an additional component comprising an adipose derived matrix.

In some embodiments, implanting a volumizing graft as described and contemplated herein at a treatment site during a plastic or reconstructive surgical procedure may provide opportunities to perform further techniques which provide autologous bioactive materials recovered from the recipient undergoing the procedure to the treatment site. More particularly, in some embodiments one or more autologous bioactive materials, such as without limitation, cells, fat or other adipose derived material, whole blood, blood components (e.g., red blood cells (RBCs), plasma, etc.), blood derivatives (e.g., PRP, etc.) and derivatives, may be injected or otherwise delivered to the cavity of the volumizing grafts or to an additional component at least partially contained therein, to the second surface of the volumizing graft which faces surrounding tissues at the treatment site. Upon delivery to the treatment site, such one or more autologous materials may infiltrate, migrate, infuse, or otherwise flow into or through, at least a portion of the sheet shaped body or an additional component which is at least partially contained in the cavity or deposited as a coating on a surface of the sheet shaped body, thereby being available to provide additional beneficial biological effects at or proximate to the treatment site.

The production of grafts having the most effective combination of physical properties necessary to achieve the intended results, which depend on the particular surgical procedure and treatment goals, can be difficult.

Some of the issues that arise during and after repair and reconstruction surgery involving use of grafts: failure to maintain the desired size, shape, volume, etc. of the graft, unintended shifting or positional movement of the graft, and failure to provide or maintain desired suppleness, rigidity, smoothness, and other qualities relating to texture, handling, feel, and appearance of the body feature.

For example, some surgical procedures are performed, at least in part, to replace mass, volume, or both, of a body feature that has been lost through disease, atrophy, excision, etc. Some surgical procedures are performed, at least in part, to provide additional mass, volume, or both, to a body feature, for enlarging or reshaping the body feature, or both. In either case, the surgical procedure may be intended to restore the health, structure, and function of a body feature, or to change the appearance, shape, or size of a body feature, or some combination of these goals.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

We claim:

1. A volumizing graft for implanting in, or, or proximate to a body feature of a subject, thereby forming a treatment site, the volumizing graft comprising:
a sheet shaped body, which has a first surface and an opposite second surface and which comprises one or more sheet shaped components, each of which comprises one or more biocompatible materials;
wherein the body forms, or is capable of forming, a three dimensional shape in which the first surface at least partially defines a cavity and the second surface at least partially forms an external surface,
wherein the cavity is sized and shaped to receive and retain therein, for at least a period of time, one or more additional components.

2. The volumizing graft of claim 1, wherein at least one of the one or more biocompatible materials is also bioactive.

3. The volumizing graft of claim 1, wherein the three dimensional shape of the sheet shaped body comprises at least a portion of one or more shapes selected from: a pouch, a sling, a balloon, a wrap, a cap, a covering, a container, a retainer, an envelope, and an irregular shape.

4. The volumizing graft of claim 1, wherein at least one of the one or more biocompatible materials of at least one of the one or more sheet shaped components comprises one or more tissue-derived matrices.

5. The volumizing graft of claim 1, wherein the sheet shaped body comprises two or more sheet shaped components which are combined, at least partially, by one or more techniques selected from: attaching, connecting, layering, arranging, and affixing, to form the sheet shaped body.

6. The volumizing graft of claim 5, wherein at least two of the two or more sheet shaped components each comprises one or more tissue-derived matrices, wherein each of the one or more tissue-derived matrices may be the same or different from one another in type of tissue, physical form, one or more other physical and bioactive characteristics, and combinations thereof.

7. The volumizing graft of claim 6, wherein each tissue derived matrix is derived from one or more tissue samples recovered from one or more donors, wherein each of the one or more tissue samples is selected from one or more of: an adipose tissue, a connective tissue, a dermis tissue, a duodenal tissue, a fascia tissue, an intestinal mucosal tissue, an intestinal serosal tissue, a membranous tissue, a muscle tissue, a pericardial tissue, a periosteal tissue, a peritoneal tissue, an amniotic tissue, a chorionic tissue, and an umbilical cord tissue.

8. The volumizing graft of claim 1, wherein the sheet shaped body include one or more attachment features which facilitate wrapping and securing the body at least partially around the one or more additional components.

9. The volumizing graft of claim 8, wherein the one or more attachment features are selected from: a tab, a strap, a ribbon, an extension, a petal, a suture, and multiples and combinations thereof, wherein the one or more attachment features are sized and shaped for wrapping around and retaining at least a portion of the one or more additional components in the cavity defined by the first surface of the three dimensional shape formed by the sheet shaped body.

10. The volumizing graft of claim 1, wherein the sheet shaped body or at least one of the one or more sheet shaped components from which the body is formed, includes one or both of:
a) one or more pass-through features which are configured to allow passage or migration of fluids, bioactive substances, cells, regenerating tissue, and other substances therethrough;
b) one or more expansion features which enable the sheet shaped body or one or more sheet shaped components to expand and cover or contain more of the one or more additional components held in the cavity, or cover or contact more of a treatment site, or a combination thereof.

11. The volumizing graft of claim 10, wherein either or both of the pass-through features and expansion features include one or more of: perforations, openings, slots, meshing, grooves, channels, each of which may or may not extend entirely through a sheet shaped body or component from a first surface to a second surface thereof.

12. The volumizing graft of claim 1, further comprising at least one additional component, which may or may not already be at least partially contained in the cavity of the sheet shaped body.

13. The volumizing graft of claim 12, wherein after implanting, the at least one additional component is at least partially contained in the cavity and the sheet shaped body affixes and maintains the at least one additional component at least partially in a desired position and orientation relative to the treatment site, either permanently or temporarily.

14. The volumizing graft of claim 12, wherein the at least one additional component has one or more physical forms selected from: monolithic, a simple or complex geometric shape, an irregular shape, particulates, pieces, fibers, and chunks.

15. The volumizing graft of claim 12, wherein the at least one additional component comprises an implant having one or more physical forms selected from:
(1) a pre-formed three dimensional shape which is capable of retaining its own shape in the absence of external support or containment;
(2) a particulate form, which is at least partially dehydrated or not; and
(3) a flowable mixture comprising a liquid, a paste, a putty, a gel, or a combination thereof.

16. The volumizing graft of claim 12, wherein one or more of the at least one additional component comprises a tissue-derived matrix produced by processing one or more tissue samples recovered from one or more donors.

17. The volumizing graft of claim 16, wherein each of the one or more tissue samples, independently of other tissue samples, comprises one or more tissue types selected from: adipose tissue, amnion tissue, artery tissue, bone tissue, breast tissue, cartilage tissue, connective tissue, chorion tissue, colon tissue, non-calcified dental tissue, dermal tissue, duodenal tissue, endothelial tissue, epithelial tissue, fascial tissue, gastrointestinal tissue, gingival tissue, growth plate tissue, intervertebral disc tissue, intestinal mucosal tissue, intestinal serosal tissue, ligament tissue, liver tissue, lung tissue, mammary tissue, membranous tissue, meniscal tissue, muscle tissue, nerve tissue, ovarian tissue, parenchymal organ tissue, pericardial tissue, periosteal tissue, peritoneal tissue, placental tissue, skin tissue, spleen tissue, stomach tissue, synovial tissue, tendon tissue, testes tissue, umbilical cord tissue, urological tissue, vascular tissue, and vein tissue.

18. The volumizing graft of claim 16, wherein each tissue-derived matrix of the one or more additional components comprising a tissue-derived matrix has, independently of other tissue-derived matrices, one or more physical forms selected from:
(1) a pre-formed three dimensional shape which is capable of retaining its own shape in the absence of external support or containment;
(2) a particulate form, which is at least partially dehydrated or not; and
(3) a flowable mixture comprising a liquid, a paste, a putty, a gel, or a combination thereof.

19. A reconstructive or cosmetic surgical method for reconstructing, reshaping, or both reconstructing and reshaping, a body feature by replacement or enhancement of tissue mass and volume, the method comprising the steps of:
selecting a treatment site in, on, or proximate to the body feature;
implanting the sheet shaped body of the volumizing graft of claim 12 in or on the treatment site, wherein the external surface of the body of the volumizing graft is at least partially in contact with host tissue at the treatment site,
either before or after implanting the volumizing graft, positioning the at least one additional component at least partially in the cavity of the sheet shaped body.

20. The surgical method of claim 19, wherein a post-operative period of time comprises a period of time after implanting the sheet shaped body and at least one additional component of the volumizing graft, the surgical method further comprising the step of:
replacing, supplementing, or both, of at least a portion of one or more of the at least one additional component at the treatment site, with at least one secondary additional component, wherein the at least one secondary additional component has characteristics which may or may not be different from those of the at least one additional component contained in the cavity of the implanted sheet shaped body.

21. The surgical method of claim 19, wherein a post-operative period of time comprises a period of time after implanting the sheet shaped body and at least one additional component of the volumizing graft, the surgical method further comprising the step of:
removing, repositioning, or both, at least a portion of one or more of the at least one additional component at the treatment site after passage of one or more post-operative periods of time.

22. The surgical method of claim 19, wherein the sheet shaped body of the volumizing graft is initially in a planar configuration, and the step of implanting the sheet shaped body comprises reshaping the sheet shaped body of the volumizing graft to form a desired three dimensional shape, based on characteristics of the body feature and treatment site in, on, or proximate thereto.

23. The surgical method of claim 19, wherein the step of positioning the at least one additional component at least partially in the cavity of the sheet shaped body is performed by inserting, placing, disposing, delivering, injecting, pouring, flowing, rolling, folding, expanding, pausing for a time sufficient for spontaneous unrolling, unfolding, or expansion to occur, or a combination thereof, based at least partially on the physical form of each of the at least one additional component and characteristics of the treatment site.

24. The surgical method of claim 19, wherein the body feature comprises a breast and the reconstructive or cosmetic surgical method comprises post-mastectomy reconstruction and augmentation or replacement of breast tissue.
